(19) United States
(12) Patent Application Publication
(10) Pub. No.: US 2026/0038634 A1
(43) Pub. Date: Feb. 5, 2026

(54) GENE MUTATION DETECTION METHOD AND APPARATUS, DEVICE, MEDIUM, AND PRODUCT

(71) Applicant: GENEMIND BIOSCIENCES COMPANY LIMITED, Shenzhen (CN)

(72) Inventors: Yixin LIN, Shenzhen (CN); Baosheng FENG, Shenzhen (CN); Qigang LI, Shenzhen (CN); Qunting LIN, Shenzhen (CN); Chaoyang LI, Shenzhen (CN)

(73) Assignee: GENEMIND BIOSCIENCES COMPANY LIMITED, Shenzhen (CN)

(21) Appl. No.: 19/288,031

(22) Filed: Aug. 1, 2025

(30) Foreign Application Priority Data
Aug. 2, 2024 (CN) 202411059743.6
Aug. 2, 2024 (CN) 202411063862.9

(51) Int. Cl. G16B 30/00; G16B 40/20

(52) U.S. Cl. CPC G16B 30/00; G16B 40/20

(57) ABSTRACT

Provided are a gene mutation detection method and apparatus, a device, a medium, and a product. The method includes acquiring a suspected mutation site of a nucleic acid sample under test, where the suspected mutation site is determined based on first mutation feature data generated by a first mutation detection module upon mutation calling performed on sequencing data of the nucleic acid sample under test, and the recall at which the first mutation detection module identifies gene mutation sites is greater than or equal to a preset recall; acquiring second mutation feature data and third mutation feature data of each suspected mutation site; and inputting the second mutation feature data and the third mutation feature data into a pre-trained target mutation detection model and outputting a mutation detection result of each suspected mutation site.

### CROSS-REFERENCE TO RELATED APPLICATIONS

This application claims priority to Chinese Patent Application No. 202411063862.9, filed on Aug. 2, 2024, and Chinese Patent Application No. 202411059743.6, filed on Aug. 2, 2024, the contents of each of which are hereby incorporated by reference.

### TECHNICAL FIELD

This invention relates to the field of biotechnology, particularly a gene mutation detection method and apparatus, a device, a medium, and a product.

### BACKGROUND OF THE INVENTION

Gene mutation detection is one of the important steps in the process of bioinformatics data processing. Due to the complexity of biological data and the influence of factors such as sequencing bias of sequencing platforms and batch variation of sequencing reagents, current gene mutation detection methods or software systems still have certain detection errors.

In particular, for mutation sites with relatively low mutation frequencies, such as mutation frequencies less than 1.5%, current gene mutation detection methods or software systems cannot ensure both the precision and the recall of gene mutation detection.

### SUMMARY

Embodiments of this invention provide a gene mutation detection method and apparatus, a device, a medium, and a product to address the problem of poor performance of traditional mutation detection software to ensure both the precision and the recall of gene mutation detection.

An embodiment of this invention provides a gene mutation detection method.

The method includes acquiring a suspected mutation site of a nucleic acid sample under test, where the suspected mutation site is determined based on first mutation feature data generated by a first mutation detection module upon mutation calling performed on sequencing data of the nucleic acid sample under test, and the recall at which the first mutation detection module identifies gene mutation sites is greater than or equal to a preset recall; performing, by a second mutation detection module, feature extraction on each suspected mutation site to obtain second mutation feature data; processing, by a sequencing data processing module, the sequencing data to obtain overall processed sequencing data and screening processed sequencing data of each suspected mutation site from the overall processed sequencing data to obtain third mutation feature data; and inputting the second mutation feature data and the third mutation feature data into a pre-trained target mutation detection model and outputting a mutation detection result of each suspected mutation site.

An embodiment of this invention provides a method for constructing simulated tumor reference standard sequencing data. The method includes (a) for a targeted region, acquiring a first germline mutation site set from a first human genome reference standard and a second germline mutation site set from a second human genome reference standard; (b) selecting, from the first germline mutation site set, a set of unique germline mutation sites relative to the second germline mutation site set; and (c) acquiring sequencing data of the second human genome reference standard and sequencing data of the first human genome reference standard, and for at least one preset simulated somatic mutation site, in accordance with a predetermined replacement ratio, replacing sequencing data originating from the at least one preset simulated somatic mutation site in the sequencing data of the second human genome reference standard with sequencing data originating from the at least one preset simulated somatic mutation site in the sequencing data of the first human genome reference standard, thereby obtaining the simulated tumor reference standard sequencing data containing simulated somatic mutations, where the at least one preset simulated somatic mutation site is selected from the set of unique germline mutation sites.

An embodiment of this invention provides a gene mutation detection apparatus. The apparatus includes a suspected mutation site acquisition module, a second mutation feature data acquisition module, a third mutation feature data acquisition module, and a mutation detection result output module.

The suspected mutation site acquisition module is configured to acquire a suspected mutation site of a nucleic acid sample under test, where the suspected mutation site is determined based on first mutation feature data generated by a first mutation detection module upon mutation calling performed on sequencing data of the nucleic acid sample under test, and the recall at which the first mutation detection module identifies gene mutation sites is greater than or equal to a preset recall.

The second mutation feature data acquisition module is configured to perform feature extraction through a second mutation detection module on each suspected mutation site to obtain second mutation feature data.

The third mutation feature data acquisition module is configured to process the sequencing data through a sequencing data processing module to obtain overall processed sequencing data and screen processed sequencing data of each suspected mutation site from the overall processed sequencing data to obtain third mutation feature data.

The mutation detection result output module is configured to input the second mutation feature data and the third mutation feature data into a pre-trained target mutation detection model and output a mutation detection result of each suspected mutation site.

An embodiment of this invention provides an electronic device. The electronic device includes at least one processor; and a memory communicatively connected to the at least one processor.

The memory stores a computer program executable by the at least one processor to enable the at least one processor to perform the gene mutation detection method of any embodiment of this invention.

An embodiment of this invention provides a computer-readable storage medium storing computer instructions which, when executed by a processor, cause the processor to perform the gene mutation detection method of any embodiment of this invention.

An embodiment of this invention provides a computer program product. The computer program product includes a computer program which, when executed by a processor, causes the processor to perform the gene mutation detection method of any embodiment of this invention.

Solutions of embodiments of this invention include acquiring a suspected mutation site of a nucleic acid sample under test by using the first mutation detection module with a recall greater than or equal to a preset recall, thereby ensuring the completeness of detecting the suspected mutation site; performing feature extraction on each suspected mutation site by using the second mutation detection module to obtain second mutation feature data; processing the sequencing data by using the sequencing data processing module to obtain overall processed sequencing data and screening processed sequencing data of each suspected mutation site from the overall processed sequencing data to obtain third mutation feature data; and inputting the second mutation feature data and the third mutation feature data into the pre-trained target mutation detection model and outputting a mutation detection result of each suspected mutation site. The target mutation detection model of this invention, based on the first mutation feature data with a high recall contribution, integrates the second mutation feature data extracted by the second mutation detection module different from the first mutation detection module and the third mutation feature data screened from sequencing data and related to suspected mutation features. The combination of the three types of mutation features addresses the problem of poor detection performance of traditional mutation detection methods or software systems and ensures both the high precision and the high recall of gene mutation detection.

It is to be understood that the content described in this part is neither intended to identify key or important features of embodiments of this invention nor intended to limit the scope of this invention. Other features of this invention are apparent from the description provided hereinafter.

### BRIEF DESCRIPTION OF THE DRAWINGS

To illustrate solutions of embodiments of this invention more clearly, drawings used in description of embodiments of this invention are described hereinafter. Apparently, these drawings illustrate part of embodiments of this invention. Those of ordinary skill in the art may obtain other drawings based on these drawings on the premise that no creative work is done.

FIG. 1 is a flowchart of a gene mutation detection method according to an embodiment of this invention.

FIG. 2 is a flowchart of a gene mutation detection method according to an embodiment of this invention.

FIG. 3 is a diagram illustrating the meaning of an average mapping quality value according to an embodiment of this invention.

FIG. 4 is a diagram of a unique germline mutation site of HG002 relative to HG001 according to an embodiment of this invention.

FIG. 5 is a diagram of a unique germline mutation site of HG002 relative to HG001 according to an embodiment of this invention.

FIG. 6 is a flowchart of a method for constructing sequencing data of simulated somatic mutations according to an embodiment of this invention.

FIG. 7 is a diagram of the result of paired simulated tumor reference standard sequencing data and simulated normal sample sequencing data according to an embodiment of this application.

FIG. 8 is a diagram of the recall evaluation result of simulated tumor reference standard sequencing data according to embodiments of this application.

FIG. 9 is a diagram of the accuracy evaluation result of simulated tumor reference standard sequencing data according to embodiments of this application.

FIG. 10 is a flowchart of a method for training an XGBoost model according to an embodiment of this invention.

FIG. 11 is a diagram illustrating the structure of a gene mutation detection apparatus according to an embodiment of this invention.

FIG. 12 is a diagram of a simulated tumor reference standard sequencing data construction module according to an embodiment of this invention.

FIG. 13 is a diagram illustrating the structure of an electronic device according to an embodiment of this invention.

### DETAILED DESCRIPTION OF THE INVENTION

For a better understanding of solutions of this invention by those skilled in the art, solutions in embodiments of this invention are described clearly and completely hereinafter in conjunction with the drawings in embodiments of this invention. Apparently, the embodiments described hereinafter are part, not all, of embodiments of this invention. Based on embodiments of this invention, all other embodiments obtained by those of ordinary skill in the art on the premise that no creative work is done are within the scope of this invention.

It is to be noted that the terms “first”, “second”, “third”, “initial”, “target”, “reference”, and the like in the description, claims and the preceding drawings of this disclosure are used for distinguishing between similar objects and are not necessarily used for describing a particular order or sequence. It is to be understood that the data used in this way is interchangeable where appropriate so that embodiments of this invention described herein may also be implemented in a sequence not illustrated or described herein. Additionally, the terms “include” and “have” and any variations thereof are intended to encompass a non-exclusive inclusion. For example, a process, method, system, product, or device that includes a series of steps or units not only includes the expressly listed steps or units but may also include other steps or units that are not expressly listed or are inherent to such process, method, product, or device.

In embodiments of this application, the term “sequencing” may also be referred to as “nucleic acid sequencing” or “gene sequencing,” and these three terms are interchangeable in expression, all referring to the determination of the type and order of bases or nucleotides (including nucleotide analogs) in a nucleic acid molecule. The sequencing includes the process of incorporating nucleotides onto a template and capturing the corresponding signals emitted from the nucleotides (including analogs). Herein there is no special limitation on the sequencing platform; it may be any commonly used first-, second-, or third-generation sequencing platform. Herein the sequencing platforms for performing the above sequencing methods may include, but are not limited to, GenoCare 1600/GenoLab M/FASTASeq 300/SURFSeq 5000 from GeneMind Biosciences, HiSeq/MiSeq/NextSeq series/NovaSeq series from Illumina, Ion Torrent from Thermo Fisher/Life Technologies, BGISEQ and MGISEQ/DNBSEQ, and single-molecule sequencing platforms. The sequencing includes sequencing by synthesis (SBS) and/or sequencing by ligation (SBL), including DNA sequencing and/or RNA sequencing. The sequencing results/data obtained from sequencing, that is, the read-out nucleic acid fragments or read sequences, are called reads.

In embodiments of this application, VarScan is a software written in Java for Linux systems, is used for performing tumor somatic mutation detection (calls SNV in somatic variants), and is applicable to various sequencing data generated from targeted sequencing, exome sequencing, and whole-genome resequencing. Strelka2 is a mutation detection software for analyzing germline variants in small cohorts and somatic variants in tumor/non-mutant paired samples. Mutect2 is a tool within GATK (The Genome Analysis Toolkit developed by the Broad Institute and used for second-generation resequencing data analysis). Mutect2 is primarily designed to detect somatic variants. Mutect2 includes SNV and InDel.

When detecting gene mutations in biological samples, traditional mutation detection software has limited performance and faces difficulties in balancing the precision and the recall of mutation detection. This deficiency in model performance is especially pronounced in biological samples with low mutation frequencies.

In this application, the term “recall” (also referred to as “recall rate”) represents the proportion of actual positive samples that are correctly predicted. Specifically, the recall is the ratio of the number of samples predicted as positive and actually positive to the total number of actual positive samples or is the probability that actual positive samples are predicted as positive by the model. By way of example, the recall can be expressed as Recall=TP/(TP+FN).

The term “precision” (also referred to as “precision rate”) represents the ratio of actually positive samples to samples predicted as positive. The precision is the ratio of the number of samples predicted as positive and actually positive to the total number of samples predicted as positive or is the probability that samples predicted as positive by the model are actually positive. By way of example, the precision can be expressed as precision=TP/(TP+FP).

TP (Truc Positive) denotes the number of instances correctly predicted by the model as positive or the number of samples predicted as positive and actually positive. FP (False Positive) denotes the number of actual negative instances wrongly predicted as positive by the model or samples predicted as positive but actually negative. FN (False Negative) denotes the number of actual positive instances wrongly predicted as negative or samples predicted as negative but actually positive. Using gene mutation detection as an example, TP represents the number of gene loci predicted by the model to have mutations and actually having mutations; FP represents the number of gene loci predicted to have mutations but actually without mutations; and FN represents the number of gene loci predicted not to have mutations but actually having mutations. In embodiments of this application, positive samples are true mutation sites, and negative samples are false positive mutation sites. The false positive mutation sites may represent variant sites caused by sequencing errors.

The term “simulated tumor reference standard sequencing data” used herein refers to a set of data artificially constructed by computational methods. This set of data mimics the sequencing results of actual tumor samples. The simulated tumor reference standard sequencing data is based on modifications or processing of existing sequencing data from cells or tissues to introduce specific variant or mutation features, thereby simulating the genomic features of tumor cells. It is to be noted that the simulated tumor reference standard sequencing data do not directly originate from real tumor sample sequencing, but are synthesized by researchers through algorithm design based on known tumor genomic features and sequencing technology features. These data include simulated somatic mutations and other tumor-related genomic features. Such simulated data can be used for evaluating and optimizing sequencing technologies and bioinformatics analysis algorithms and workflows as well as for training and testing machine learning models.

The following description uses biological samples with mutation frequencies ranging from 0.5% to 1.5% as the research object.

When using Strelka 2 and Mutect 2 for gene mutation detection, the recall reaches a plateau as the sequencing depth (Read Depth (DP)) increases. At this point, even increasing the sequencing depth (Read Depth (DP)) further does not significantly improve the recall. This plateau depends on the sequencing platform, especially on the sequencing error rate. By way of example, the recall on the Element AVITI platform is about 0.85 while the recall on the NovaSeq 6000 platform is about 0.75. The precision of gene mutation detection is determined by the sequencing platform and generally maintains a high precision above 0.9.

When using VarScan for gene mutation detection, compared with Strelka 2 and Mutect 2, the recall can reach a high value of about 0.96 as the sequencing depth (Read Depth (DP)) increases. However, VarScan has the disadvantage of very low precision. In practical applications, to improve the precision, additional genomic annotation, genetic annotation, and manual review are required after mutation detection to identify true mutations. The lack of automation makes it extremely time- and labor-intensive.

Thus, when facing somatic mutations with mutation frequencies below 1.5%, neither screening the sequencing platform nor increasing the sequencing depth (Read Depth (DP)) alone can achieve both a high recall and a high precision.

In view of this, embodiments of this invention provide a gene mutation detection method and apparatus, a device, a medium, and a product that can achieve better gene mutation detection results, capable of balancing high precision and high recall even for gene mutations with frequencies less than or equal to 1.5%.

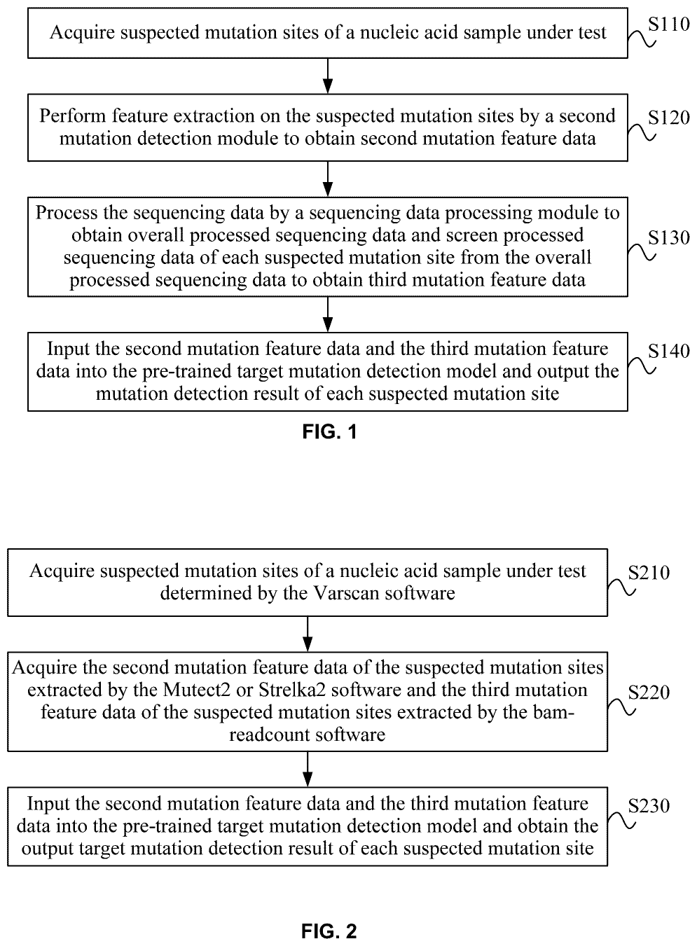

FIG. 1 is a flowchart of a gene mutation detection method according to an embodiment of this invention. This embodiment is applicable to mutation detection on variant sites in sequencing data. The method can be performed by a gene mutation detection apparatus. The gene mutation detection apparatus may be implemented in hardware and/or software and configured in a terminal device. As shown in FIG. 1, the method includes the following:

In S110, suspected mutation sites of a nucleic acid sample under test are acquired.

Specifically, the nucleic acid sample under test represents a sample containing nucleic acid molecules or nucleic acid mixtures and used for mutation detection. The nucleic acid mixture includes two or more types of nucleic acid molecules having different sequences. In embodiments of this invention, the term “nucleic acid molecule” refers to a polymer of nucleotides of any length. The nucleic acid molecule may be a short nucleic acid fragment less than or equal to 500 bp, for example, 20 bp, 50 bp, 80 bp, 100 bp, 120 bp, 150 bp, 180 bp, 200 bp, 220 bp, 250 bp, 280 bp, 300 bp, 320 bp, 350 bp, 380 bp, 400 bp, 420 bp, 450 bp, 480 bp, or 500 bp, or may be a long nucleic acid fragment longer than 500 bp. In embodiments of this invention, the “nucleic acid molecule” includes a nucleic acid molecule composed of ribonucleotides or a nucleic acid molecule composed of deoxyribonucleotides. Moreover, the nucleic acid sample under test in embodiments of this application may be an amplified nucleic acid sample. That is, for each nucleic acid molecule sequence in the nucleic acid sample under test, there are multiple copies of that nucleic acid molecule.

By way of example, the sample sources include, but are not limited to, blood, urine, plasma, cell samples, ex vivo tissue fluid, or tumor tissue. Here the sample sources of the nucleic acid sample under test are not limited. In an embodiment, the sample is a nucleic acid sample under test derived from humans. That is, the gene mutation detection method of embodiments of this application is a gene mutation detection method for human samples. It is to be understood that this method is also applicable to gene mutation detection of nucleic acid sample under tests derived from other biological species. In the following description, human-derived nucleic acid sample under tests are used as examples but are not intended to limit the species source of the nucleic acid sample under tests.

In some embodiments, the nucleic acid sample under test has a mutation frequency less than or equal to 1.5%. Specifically, the mutation frequency refers to, for a sample, at a particular genomic locus, the ratio of the sequencing depth (Read Depth (DP)) of a specific mutation to the total sequencing depth (Read Depth (DP)). For example, in the reference genome, if the total number of bases at genomic locus P is 100 (that is, the sequencing depth (Read Depth (DP)) is 100) and the base is adenine A (meaning that without mutation, the base detected at the genomic locus P is theoretically adenine A). In the gene sequencing data of the nucleic acid sample under test, the genomic locus P has a cytosine (C) mutation with 5 bases (that is, the sequencing depth (Read Depth (DP)) is 5), in other words, five adenine (A) bases at genomic locus P are mutated to cytosine (C), then the gene mutation frequency in the nucleic acid sample under test is 5%.

Traditional gene mutation detection software performs poorly on nucleic acid samples with mutation frequencies less than or equal to 1.5%, failing to ensure both high precision and high recall. The target mutation detection model in this embodiment is especially suitable for mutation detection of nucleic acid samples with mutation frequencies less than or equal to 1.5%, capable of balancing high precision and high recall of detection results.

In some embodiments, the nucleic acid sample under test is a biological sample with somatic mutations or a biological sample with germline mutations. Somatic mutations refer to mutations arising in somatic cells during individual development due to mutagenic factors, which generally are not inherited by offspring. Germline mutations refer to mutations carried by germ cells during sexual reproduction, which can be inherited by offspring.

Specifically, the sequencing data of the nucleic acid sample under test represents nucleic acid sequence obtained by sequencing the sample using sequencing technology. For example, sequencing technologies include, but are not limited to, Sanger sequencing, high-throughput sequencing, and single-molecule sequencing. Sequencing technologies are not limited here and can be configured as required. Sequencing may be single-end or paired-end. Sequencing can be performed on sequencing platforms including, but not limited to, Illumina's Hiseq/Miseq/Nextseq/Novaseq platform, Thermo Fisher/Life Technologies' Ion Torrent platform, BGI's BGISEQ and MGISEQ platforms, single-molecule sequencing platforms, and GeneMind's GenoLab M/GenoCare 1600/FASTASeq 300/SURFSeq 5000 platform.

In embodiments of this application, the mutation site refers to a genomic locus in the nucleic acid sample where a base mutation causes the sequence to differ from the reference genome. It is to be understood that in embodiments of this application, mutation sites belong to the category of variant sites, with the ratio of mutation sites to variant sites ranging between 0 and 1.

In embodiments of this application, the suspected mutation sites are determined by first mutation feature data obtained from mutation detection of a first mutation detection module on sequencing data of the nucleic acid sample under test. Analyzing the sequencing data of the nucleic acid sample under test with the first mutation detection module can extract mutation features inherently contained in the sequencing data and/or mutation features obtained after further data processing of the sequencing data. In embodiments of this application, the mutation feature data obtained by the first mutation detection module is referred to as first mutation feature data. That is, in embodiments of this application, the suspected mutation sites are determined by first mutation feature data obtained from mutation detection of a first mutation detection module on sequencing data of the nucleic acid sample under test.

In embodiments of this application, when the first mutation detection module for mutation detection of the nucleic acid sample under test identifies gene mutation sites, that is, performs mutation detection, the recall is greater than or equal to a preset recall, that is, the recall in identifying gene mutation sites is greater than or equal to a preset recall, ensuring that the probability of identifying true mutation sites satisfies a preset probability requirement. The suspected mutation sites obtained by the first mutation detection module are then passed to step S120, where a second mutation detection module satisfying a preset precision requirement further analyzes them. The second mutation feature data extracted by the second mutation detection module is used as one of the input features of the target mutation detection model. This allows the target mutation detection model in step S140 to output mutation detection results balancing high recall and high precision.

In some embodiments, the preset recall is 0.9, meaning the first mutation detection module's recall in identifying gene mutation sites or in mutation detection is greater than or equal to 0.9, thereby reducing the probability of missing true mutation sites. In some embodiments, the first mutation detection module's recall in identifying gene mutation sites is greater than or equal to 0.95.

In some embodiments, acquiring the suspected mutation site of the nucleic acid sample under test includes aligning, by an alignment unit of the first mutation detection module, input sequencing data of the nucleic acid sample under test with sequencing data of a reference genome to obtain at least one gene variant site in the sequencing data of the nucleic acid sample different from the sequencing data of the reference genome; performing, by a feature extraction unit of the first mutation detection module, feature extraction on the at least one gene variant site to obtain at least one piece of first mutation feature data; and screening, by a mutation detection unit of the first mutation detection module, the suspected mutation site from the at least one gene variant site based on the at least one piece of first mutation feature data.

Due to the high recall of the first mutation detection module, this configuration ensures the completeness of the identified suspected mutation sites and makes certain false-positive mutation sites filtered out, thereby reducing the data amount of the suspected mutation sites and improving the gene mutation detection efficiency.

For the sequencing data of the nucleic acid sample under test, variant sites represent genomic positions that differ from the reference genome and are present in the sequencing data. By way of example, the variant types of the suspected mutation sites include, but are not limited to, single nucleotide variation (SNV), insertion and deletion mutations, mismatch mutations, and tandem repeats. Single nucleotide polymorphism emphasizes the mutation frequency of suspected mutation sites in the population (for example, the mutation frequency >1%), where a single base at the suspected mutation site differs from the corresponding base in the reference genome. Single nucleotide variation does not emphasize the mutation frequency of suspected mutation sites in the population, where a single base at the suspected mutation site differs from the corresponding base in the reference genome. Insertion or deletion mutation represents the presence or absence of a new nucleic acid sequence fragment different from the reference genome and at the gene mutation site. Mismatch mutation represents the presence of mismatched base pairs at the gene mutation site. Tandem repeat represents the presence of one or more repeated nucleotides at the suspected mutation site.

Variant sites can be obtained by aligning sequencing data using a sequencing data alignment tool. By way of example, the alignment tool may be Bowtie2 or BLAST. Here the alignment tool is not limited and can be configured based on actual requirements.

Mutation sites refer to gene loci in a nucleic acid sample where base mutations cause sequence discrepancies with the reference genome, including single nucleotide variation and polynucleotide variant. It is to be understood that in embodiments of this application, mutation sites belong to the category of variant sites, with the ratio of mutation sites to variant sites ranging between 0 and 1.

In S120, feature extraction is performed on the suspected mutation sites by a second mutation detection module to obtain second mutation feature data.

To distinguish the mutation feature data extracted by the second mutation detection module from the first mutation feature data extracted by the first mutation detection module, the mutation feature data extracted by the second mutation detection module is referred to as “second mutation feature data”.

In embodiments of this application, both the first and second mutation detection modules are models used for detecting mutations in nucleic acid samples, but they operate on different mutation detection principles. In embodiments of this application, the second mutation detection module is introduced, and the mutation detection function of the second mutation detection module is used to extract features from the suspected mutation sites identified by the first mutation detection module. This is done to improve the precision of the target mutation detection model-trained using the extracted features as part of the features-in identifying the gene mutation sites, thereby ensuring that the proportion of true mutation sites among the suspected mutation sites identified by the first mutation detection module satisfies a predefined proportion.

In some embodiments, the precision of the second mutation detection module is greater than or equal to a preset precision, and the preset precision ensures that the precision of the target mutation detection model is greater than or equal to 0.8. By configuring the first mutation detection module to satisfy the preset recall, it is possible to improve the probability of identifying true mutation sites. Subsequently, the suspected mutation sites from the first mutation detection module are further analyzed by the second mutation detection module that satisfies the preset precision, and the extracted second mutation features are used as part of the input to the target mutation detection model, allowing the mutation detection results output by the target mutation detection model in step S140 to achieve both a high recall and a high precision.

In some embodiments, the preset precision is 0.9, meaning the second mutation detection module's precision in identifying gene mutation sites or in mutation detection is greater than or equal to 0.9. In this case, when identifying the gene mutation sites, the first mutation detection module has a recall greater than or equal to the preset recall, and the second mutation detection module has a precision greater than or equal to the preset precision. By configuring the first mutation detection module to satisfy the preset recall, it is possible to improve the probability of identifying true mutation sites. Subsequently, the suspected mutation sites from the first mutation detection module are further analyzed by the second mutation detection module that satisfies the preset precision, and the extracted second mutation features are used as part of the input to the target mutation detection model, allowing the mutation detection results output by the target mutation detection model in step S140 to achieve both a high recall and a high precision.

In some embodiments, the recall of the first mutation detection module in identifying the gene mutation sites is greater than or equal to 0.9, and the precision of the second mutation detection module in identifying the gene mutation sites is also greater than or equal to 0.9. By using the first mutation detection module to reduce the probability of missing true mutation sites, it is possible to improve the likelihood of identifying the suspected mutation sites. Subsequently, the suspected mutation sites from the first mutation detection module are further analyzed by the second mutation detection module, thereby reducing the probability of missing false-positive mutation sites. Finally, the second mutation feature data extracted by the second mutation detection module are used as part of the input to the target mutation detection model, allowing the mutation detection results output by the target mutation detection model in step S140 to achieve both a high recall and a high precision.

In embodiments of this application, the recall and the precision may be obtained by testing the first mutation detection module and the second mutation detection module through a test set. The test set includes test sequencing data for testing nucleic acid samples.

In an embodiment, the first and second mutation detection modules may be screened from existing gene mutation detection software. By way of example, the first mutation detection module is implemented using the Varscan software, and the second mutation detection module is implemented using the Mutect2 software. In this case, when identifying gene mutation sites, the VarScan software exhibits a high recall, reaching up to 0.96, thereby increasing the probability of identifying true mutation sites. The suspected mutation sites obtained by VarScan are further analyzed using the Mutect2 software that has a precision greater than or equal to 0.9. The extracted second mutation features, together with the third mutation feature data extracted from the sequencing data based on the suspected mutation sites during sequencing data processing, are used as part of the input to the target mutation detection model. This enables the mutation detection results output by the target mutation detection model in step S140 to achieve both a high recall and a high precision. The results show that the target mutation detection model exhibits both a high recall and a high precision in mutation site detection.

In S130, the sequencing data is processed by a sequencing data processing module to obtain overall processed sequencing data, and processed sequencing data of each suspected mutation site is screened from the overall processed sequencing data to obtain third mutation feature data.

In some embodiments, screening the processed sequencing data of each suspected mutation site from the overall processed sequencing data to obtain the third mutation feature data includes, for each suspected mutation site, acquiring the processed sequencing data corresponding to the suspected mutation site from the total processed sequencing data and adding the processed sequencing data to the third mutation feature data.

In some embodiments, screening the processed sequencing data of each suspected mutation site from the overall processed sequencing data to obtain the third mutation feature data includes, for each suspected mutation site, acquiring the processed sequencing data corresponding to the suspected mutation site from the total processed sequencing data, performing feature screening on the processed sequencing data, and adding the screened processed sequencing data to the third mutation feature data.

In S140, the second mutation feature data and the third mutation feature data are input into the pre-trained target mutation detection model, and the mutation detection result of each suspected mutation site is output.

In embodiments of this application, the second and third mutation feature data are as described above.

In embodiments of this application, the representation form of the mutation detection result may be, for example, a positive site or not, a grade, a score, or a probability. The grade may be a true mutation grade or a false-positive mutation grade. The score may be a true mutation score or a false-positive mutation score. The probability may be a true mutation rate or a false-positive mutation rate. Using the true mutation grade, true mutation score, or true mutation rate as an example, the higher the true mutation grade, true mutation score, or true mutation rate, the greater the likelihood that the suspected mutation site is a true mutation site. Conversely, the lower the true mutation grade, true mutation score, or true mutation rate, the lower the likelihood that the suspected mutation site is a true mutation site.

The representation form of the target mutation detection result is not limited and may be customized based on actual requirements.

In an optional embodiment, the mutation detection result indicates that the suspected mutation site is a positive site.

In an optional embodiment, the mutation detection result indicates that the suspected mutation site is a negative site.

The result of a positive site can be confirmed based on indicators such as the true mutation grade, true mutation score, and true mutation rate, but the indicators for confirming a suspected mutation site as a positive site are not limited thereto. Similarly, the result of a negative site can be confirmed based on indicators such as the false-positive mutation grade, false-positive mutation score, and false-positive mutation rate, but the indicators for confirming a suspected mutation site as a negative site are not limited thereto.

In some embodiments, inputting the second mutation feature data and the third mutation feature data into the pre-trained target mutation detection model and outputting the mutation detection result of each suspected mutation site includes inputting the second mutation feature data and the third mutation feature data into the pre-trained target mutation detection model to obtain a predicted value indicating that the suspected mutation site is positive; aligning the predicted value with a preset target value and outputting an alignment result; if the predicted value is greater than the preset target value, outputting the result that the suspected mutation site is positive; and if the predicted value is less than the preset target value, outputting the result that the suspected mutation site is negative.

Solutions of embodiments of this invention include acquiring a suspected mutation site of a nucleic acid sample under test by using the first mutation detection module with a recall greater than or equal to a preset recall, thereby ensuring the completeness of detecting the suspected mutation site; performing feature extraction on each suspected mutation site by using the second mutation detection module to obtain second mutation feature data; processing the sequencing data by using the sequencing data processing module to obtain overall processed sequencing data and screening processed sequencing data of each suspected mutation site from the overall processed sequencing data to obtain third mutation feature data; and inputting the second mutation feature data and the third mutation feature data into the pre-trained target mutation detection model and outputting a mutation detection result of each suspected mutation site. The target mutation detection model of this invention, based on the first mutation feature data with a high recall contribution, integrates the second mutation feature data extracted by the second mutation detection module different from the first mutation detection module and the third mutation feature data screened from sequencing data and related to suspected mutation features. The combination of the three types of mutation features addresses the problem of poor detection performance of traditional mutation detection methods or software systems and ensures both the high precision and the high recall of gene mutation detection.

FIG. 2 is a flowchart of a gene mutation detection method according to an embodiment of the present invention. In this embodiment, the first mutation detection module is implemented using the VarScan software, the second mutation detection module is implemented using the Mutect2 software or the Strelka2 software, and the sequencing data processing module is implemented using the bam-readcount software. As shown in FIG. 2, the method includes the following steps:

In S210, suspected mutation sites of a nucleic acid sample under test determined by the Varscan software are acquired.

In this embodiment, the nucleic acid sample under test is from somatic cells. The suspected mutation site is determined based on first mutation feature data generated by the Varscan software upon mutation calling performed on sequencing data of the nucleic acid sample under test.

VarScan is a Java-based software used under Linux and used for detecting somatic mutations. With increasing sequencing depth (Read Depth (DP)), the recall of VarScan can reach approximately 0.96. However, after mutation detection using the VarScan software, it is still necessary to rely on methods such as genomic annotation, genetic annotation, and manual judgment to further validate the mutation detection results output by VarScan.

In S220, the second mutation feature data of the suspected mutation sites extracted by the Mutect2 or Strelka2 software and the third mutation feature data of the suspected mutation sites extracted by the bam-readcount software are acquired.

In step S220, the second mutation feature data is obtained from feature extraction performed by Mutect2 or Strelka2 on the suspected mutation sites determined by VarScan. Mutect2 or Strelka2 is software for somatic mutation detection. Mutect2 employs a Bayesian algorithm based on a Hidden Markov Model and is primarily used to detect somatic single nucleotide variants and insertion and deletion mutations. Strelka2 performs rearrangement of gene variant sites of insertion and deletion mutations and conducts mutation detection based on a Bayesian probabilistic model and the mutation feature data of the rearranged gene variant sites. As the sequencing depth increases, the recall of Mutect2 or Strelka2 reaches a plateau value. This plateau value is influenced by the average error rate of the sequencing platform to which the sequencing technology belongs; therefore the plateau value is not very high. However, the precision of Mutect2 or Strelka2 can exceed 0.9.

In some embodiments, the second mutation feature data includes at least one of sequencing depth (Read Depth (DP)), number of mutation events (Number of Events (ECNT)), quality score of germline mutation (Germline Quality (GERMQ)), median quality score of reference bases (Median Base Quality (MBQ)), median quality score of mutant bases (Median Base Quality (MBQ)), median insert fragment length of reference bases (Median Fragment Length by Allele (MFRL)), median insert fragment length of mutant bases (Median Fragment Length by Allele (MFRL)), median mapping quality score of reference bases (Median Mapping Quality (MMQ)), median mapping quality score of mutant bases (Median Mapping Quality (MMQ)), Median Position (MPOS), Negative log 10 odds of artifact in normal with same allele fraction as tumor (Negative Allele Fraction in Normal (NALOD)), Normal log 10 likelihood ratio of diploid het or hom alt genotypes (Normal Log Odds (NLOD)), or Log 10 likelihood ratio score of variant existing versus not existing (Tumor Log Odds (TLOD)).

In some embodiments, the second mutation feature data includes at least one of sequencing depth (Read Depth (DP)), number of mutation events (Number of Events (ECNT)), median quality score of reference bases (Median Base Quality (MBQ)), median quality score of mutant bases (Median Base Quality (MBQ)), median insert fragment length of reference bases (Median Fragment Length by Allele (MFRL)), median insert fragment length of mutant bases (Median Fragment Length by Allele (MFRL)), median mapping quality score of reference bases (Median Mapping Quality (MMQ)), median mapping quality score of mutant bases (Median Mapping Quality (MMQ)), Median Position (MPOS), Negative log 10 odds of artifact in normal with same allele fraction as tumor (Negative Allele Fraction in Normal (NALOD)), Normal log 10 likelihood ratio of diploid het or hom alt genotypes (Normal Log Odds (NLOD)), or Log 10 likelihood ratio score of variant existing versus not existing (Tumor Log Odds (TLOD)).

In some embodiments, the second mutation feature data includes sequencing depth (Read Depth (DP)), number of mutation events (Number of Events (ECNT)), median quality score of reference bases (Median Base Quality (MBQ)), median quality score of mutant bases (Median Base Quality (MBQ)), median insert fragment length of reference bases (Median Fragment Length by Allele (MFRL)), median insert fragment length of mutant bases (Median Fragment Length by Allele (MFRL)), median mapping quality score of reference bases (Median Mapping Quality (MMQ)), median mapping quality score of mutant bases (Median Mapping Quality (MMQ)), Median Position (MPOS), Negative log 10 odds of artifact in normal with same allele fraction as tumor (Negative Allele Fraction in Normal (NALOD)), Normal log 10 likelihood ratio of diploid het or hom alt genotypes (Normal Log Odds (NLOD)), and Log 10 likelihood ratio score of variant existing versus not existing (Tumor Log Odds (TLOD)).

The sequencing depth (Read Depth (DP)) represents the number of times a site is covered by reads. By way of example, DP=4656 represents that the site is covered by 4656 reads in the obtained gene sequencing data.

The number of mutation events (Number of Events (ECNT)) represents the number of observed variant events at an identified suspected mutation site. The variant event refers to any situation that causes bases in the reads to differ from the standard base in the human reference genome, including, but not limited to, variants caused by gene insertion, gene deletion, and gene mutation.

The quality score of germline mutation (Germline Quality (GERMQ)) represents a quality score at which an identified suspected mutation site is not a germline variant and indicates the probability that the suspected mutation site is not a germline variant. The higher the quality score of germline mutation, the higher the probability that the suspected mutation site is not a germline variant. By way of example, GERMQ=93 represents that the probability that the suspected mutation site is not a germline variant is high.

The median quality score of reference bases (Median Base Quality (MBQ)) represents a median quality score of bases that match a reference genome base at an identified suspected mutation site. In some examples, the median quality score of reference bases (Median Base Quality (MBQ)) includes the base median quality value of the alternate allele (ALT) corresponding to the suspected mutation site in reads. In some examples, the base median quality value (Median Base Quality (MBQ)) includes the base median quality value of the reference allele (REF) corresponding to the suspected mutation site in the reference genome. In some examples, the base median quality value (Median Base Quality (MBQ)) includes the base median quality value of the alternate allele (ALT) corresponding to the suspected mutation site in reads and the base median quality value of the reference allele (REF) corresponding to the suspected mutation site in the reference genome. By way of example, MBQ=20, 20 represents that the base median quality value (Median Base Quality (MBQ)) of both the alternate allele (ALT) and the reference allele (REF) at that site is 20.

The median quality score of mutant bases (Median Base Quality (MBQ)) represents the median quality value of mutant bases corresponding to an identified suspected mutation.

The median insert fragment length of reference bases (Median Fragment Length by Allele (MFRL)) represents a median insert fragment length of paired-end reads whose bases, at a genomic position corresponding to an identified suspected mutation site, match a reference genome base and thus represent an unmutated allele. In some examples, the Median Fragment Length includes the Median Fragment Length of the reference allele (REF) corresponding to the suspected mutation site in the reference genome. In some examples, the Median Fragment Length includes the Median Fragment Length of the alternate allele (ALT) corresponding to the suspected mutation site in reads and the Median Fragment Length of the reference allele (REF) corresponding to the suspected mutation site in the reference genome. By way of example, MFRL=199, 191 represents that the Median Fragment Length of the alternate allele (ALT) and the Median Fragment Length of the reference allele (REF) are 199 and 191 respectively.

The median insert fragment length of mutant bases (Median Fragment Length by Allele (MFRL)) represents a median insert fragment length of paired-end reads whose bases, at a site corresponding to an identified suspected mutation, exhibit a same mutation type and thus constitute a mutant allele.

The median mapping quality score of reference bases (Median Mapping Quality (MMQ)) represents the median mapping quality value of bases that match a reference genome base and correspond to an alternate allele of an identified suspected mutation site. In some examples, the Median Mapping Quality includes the Median Mapping Quality of the alternate allele (ALT) corresponding to the suspected mutation site in reads. In some examples, the Median Mapping Quality includes the Median Mapping Quality of the reference allele (REF) corresponding to the suspected mutation site in the reference genome. In some examples, the Median Mapping Quality includes the Median Mapping Quality of the alternate allele (ALT) corresponding to the suspected mutation site in reads and the Median Mapping Quality of the reference allele (REF) corresponding to the suspected mutation site in the reference genome. By way of example, MMQ=60, 60 represents that the Median Mapping Quality of both the alternate allele (ALT) and the reference allele (REF) is 60.

The median mapping quality score of mutant bases (Median Mapping Quality (MMQ)) represents a median mapping quality value of mutant bases corresponding to an identified suspected mutation.

The Median Position (MPOS) represents the median position from an identified suspected mutation site to the ends of reads containing the identified suspected mutation site. By way of example, MPOS=34 represents that the Median Position (MPOS) from the suspected mutation site to the starts of the reads is 34.

The Negative log 10 odds of artifact in normal with same allele fraction as tumor (Negative Allele Fraction in Normal (NALOD)) represents the negative logarithm of the probability that a mutation identical to an identified suspected mutation, with the same frequency, and in sequencing data of a non-mutant sample is a false positive. In some applications, NALOD represents the negative logarithm of the probability that a mutation in a non-mutant sample and with the same frequency as tumor is a false positive. By way of example, NALOD=1.94 represents that no suspected mutation site is found in the sequencing data of the non-mutant sample.

The Normal log 10 likelihood ratio of diploid het or hom alt genotypes (Normal Log Odds (NLOD)) represents the logarithm of the likelihood ratio that an identified suspected mutation site in sequencing data of a non-mutant sample is heterozygous or homozygous. In some examples, NLOD is used to assess the probability that the mutation in the non-mutant sample is a true germline mutation (heterozygous or homozygous) to help determine whether the mutation in the tumor sample is a somatic mutation. Since germline mutations can be either diploid heterozygous or homozygous, either case qualifies as a germline mutation. A smaller NLOD value represents a lower probability that the mutation in the non-mutant sample is a germline mutation and thus a higher probability that the mutation in the tumor sample is a somatic mutation. A lower NLOD represents a higher possibility that the suspected mutation site is a disease variant site. By way of example, NLOD=47.72 represents a very low probability that the suspected mutation site in the sequencing data of the non-mutant sample is a heterozygous or homozygous variant.

The Log 10 likelihood ratio score of variant existing versus not existing (Tumor Log Odds (TLOD)) represents the logarithm of the likelihood ratio that a suspected mutation site is a true somatic mutation and indicates the probability that the suspected mutation site is present in abnormal sequence data. In some applications, TLOD is used to directly assess the probability that a mutation detected in a tumor sample is a somatic mutation. A higher TLOD value indicates a greater probability of a true somatic mutation. By way of example, TLOD=553.69 is a very high score, indicating a high probability that a variant is present in the disease sample.

NALOD and NLOD can indirectly assess the probability that a mutation is a true somatic mutation while TLOD can directly assess this probability.

The bam-readcount software processes genomic sequencing data to obtain a readcount file. The third mutation feature data in the readcount file provides comprehensive sequencing and alignment information for suspected mutation sites, serving as critical feature evidence for gene mutation detection of suspected mutation sites.

In some optional embodiments, the third mutation feature data includes at least one of average mapping quality value (avg_mapping_quality (amq)), average base quality value (avg_basequality (ab)), average position as fraction (avg_pos_as_fraction (apf)), average number of mismatch bases as fraction (avg_num_mismatches_as_fraction (anmf)), or average sum of quality score of mismatch bases (avg_sum_mismatch_qualitie (asmq)).

In some embodiments, the third mutation feature data includes at least one of average mapping quality value (avg_mapping_quality (amq)), average base quality value (avg_basequality (ab)), or average position as fraction (avg_pos_as_fraction (apf)).

In some embodiments, the third mutation feature data includes average mapping quality value (avg_mapping_quality (amq)), average base quality value (avg_basequality (ab)), and average position as fraction (avg_pos_as_fraction (apf)).

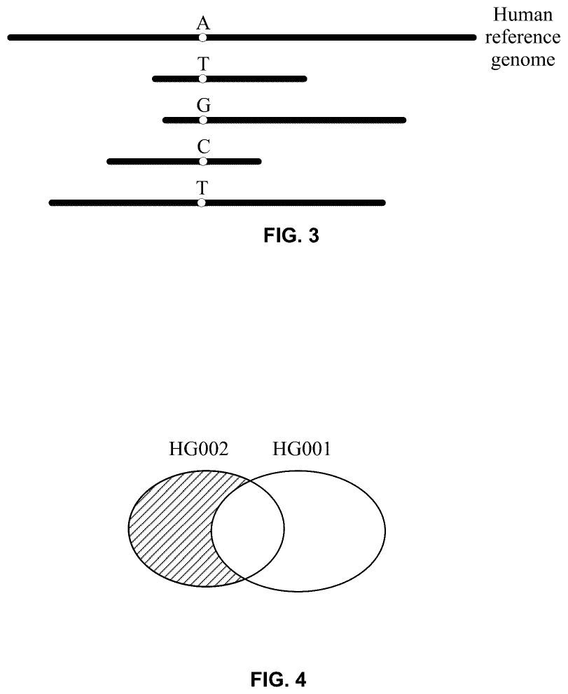

The average mapping quality value (avg_mapping_quality (amq)) is the average of mapping quality values of all detected mutant bases corresponding to a determined gene locus in a reference genome. The mapping quality value is an index used to assess how well a base at a gene variant site matches the reference genome. By way of example, using a human nucleic acid sample as an example, referring to FIG. 3, for a defined gene locus of the human reference genome (with adenine A as the type of the base), multiple nucleic acid fragments are detected to contain gene variants corresponding to this gene locus (for example, as shown in FIG. 3, the variants are thymine T, guanine G, cytosine C, and thymine T in sequence). The mapping quality value refers to the confidence that the gene mutation (thymine T, guanine G, cytosine C, and thymine T) in each nucleic acid fragment is a gene mutation corresponding to the defined gene locus (adenine A) in the human reference genome. The mapping quality value typically ranges from 0 to 40. A higher mapping quality value indicates greater reliability in aligning the base to the defined gene locus in the human reference genome.

The average base quality value (avg_basequality (ab)) is the average of base quality values of bases corresponding to an identified suspected mutation site across reads. The base quality value is an index used to assess the sequencing precision of a single base and is usually denoted by Q. The base quality value can be calculated using the formula Q=−10*log 10(E). Here E is the base calling error rate, which can be derived from the sequencing platform. A higher base quality value represents a higher sequencing precision of the base.

The average position as fraction (avg_pos_as_fraction (apf)) is the average position as fraction of base positions at a suspected mutation site relative to nucleic acid fragment reference base positions among reads containing the same suspected mutation site. For a to-be-sequenced nucleic acid molecule, when the number of sequencing cycles reaches a certain level, the sequencing performance gradually declines with further increases in the number of sequencing cycles. Therefore, the average position as fraction (avg_pos_as_fraction, apf) can be used to reflect the relative position variation of the suspected mutation site across different reads. The reference base position can be set manually. By way of example, the reference base position may be set as the position of the central base in the read, that is, the center position. By way of example, the position fraction of the center of the read is assigned a value of 1 (indicating that the variant event occurs in the middle of the read). The position fraction at the end of the read approaches 0. This is because the variant event occurs at the end of the read, and the sequencing performance deteriorates compared with bases located earlier in the read. The average position as fraction (avg_pos_as_fraction, apf) helps identify the bias pattern in the sequencing data. For example, if most gene variants are concentrated at the end of the read, it is indicated that problems exist in the sequencing or analysis process.

The average number of mismatch bases as fraction (avg_num_mismatches_as_fraction (anmf)) is an average number fraction (also referred to as average mismatch number) of bases different from a reference genome (or suspected mutation sites) across reads. The number fraction is the ratio of the number of bases in the read that do not match the reference genome to the total number of bases in the read. The number fraction represents the degree of mismatch between the gene sequencing data and the reference genome, where the total number of bases can also be understood as the length of the read. A higher average number of mismatch bases as fraction indicates a greater extent of mismatch of a gene variant site.

The average sum of quality score of mismatch bases (avg_sum_mismatch_qualitie (asmq)) is the average quality value of bases different from a human reference genome across reads corresponding to an identified suspected mutation site. The mismatch quality value is an index used to assess the quality of an individual mismatched base. A higher total corresponding to the average sum of quality score of mismatch bases indicates greater uncertainty of the suspected mutation site.

The bam-readcount software processes genomic sequencing data to obtain a readcount file. The third mutation feature data in the readcount file provides comprehensive sequencing and alignment information for suspected mutation sites, serving as critical feature evidence for gene mutation detection of suspected mutation sites.

In S230, the second mutation feature data and the third mutation feature data are input into the pre-trained target mutation detection model, and the output target mutation detection result of each suspected mutation site is obtained.

Step S230 in this embodiment corresponds to or is similar to step S140 in the embodiment shown in FIG. 1 and is not be repeated here.

In the technical solution of this embodiment, the first mutation detection module is implemented using the Varscan software, and the second mutation detection module is implemented using the Mutect2 or Strelka2 software, where Varscan satisfies the constraint of a recall ≥0.9, and Mutect2 satisfies the constraint of a precision ≥0.9. This improves both the recall and precision of the target mutation detection model.

Based on the preceding embodiments, the target mutation detection model in the embodiments of this application is pre-trained. In some embodiments, a method for training the target mutation detection model includes the steps below.

(1) The first mutation detection module acquires a training mutation site of a training nucleic acid sample.

In step (1), the training nucleic acid sample is a nucleic acid sample containing a known gene mutation site, such as a standard nucleic acid sample. That the gene mutation site is known includes that nucleic acid sequences of the training nucleic acid sample are known, and the gene mutation site can be determined according to the known sequences or includes that for a gene mutation site in the training nucleic acid sample, a position of the site in a nucleic acid sequence and a mutation type of the site can be determined. The training nucleic acid sample may contain one known gene mutation site or contain two or more known gene mutation sites.

Generally, an increase in the number of training nucleic acid samples facilitates an improvement in the accuracy of the model during training of an initial mutation detection model. Nucleic acid samples with known gene mutation sites are difficult to collect in practice. Therefore, in some implementations, the nucleic acid sample with the gene mutation site, that is, the training nucleic acid sample, may be simulated based on reference standards.

As an embodiment, the training nucleic acid sample in the embodiments of this application is a simulated tumor reference standard. Correspondingly, training sequencing data is sequencing data of the simulated tumor reference standard, that is, simulated tumor reference standard sequencing data. The simulated tumor reference standard sequencing data in the embodiments of this application is a set of data artificially constructed by computational methods. This set of data stimulates a sequencing result of an actual tumor sample. The simulated tumor reference standard sequencing data is based on modifications or processing of existing sequencing data from cells or tissues to introduce specific variant or mutation features, thereby simulating the genomic features of tumor cells. It is to be noted that the simulated tumor reference standard sequencing data do not directly originate from real tumor sample sequencing, but are synthesized by researchers through algorithm design according to known tumor genomic features and the characteristics of sequencing technology. These data include simulated somatic mutations and other tumor-associated genomic features. Such simulated data can be used for evaluating and optimizing the sequencing technology and bioinformatics analysis algorithms and workflows and training and testing machine learning models.

The embodiments of this application further provide a method for constructing the simulated tumor reference standard sequencing data. The method includes: (a) for a targeted region, acquiring a first germline mutation site set from a first human genome reference standard and a second germline mutation site set from a second human genome reference standard; (b) selecting, from the first germline mutation site set, a set of unique germline mutation sites relative to the second germline mutation site set; and (c) acquiring sequencing data of the second human genome reference standard and sequencing data of the first human genome reference standard, and for at least one preset simulated somatic mutation site, in accordance with a predetermined replacement ratio, replacing sequencing data originating from the at least one preset simulated somatic mutation site in the sequencing data of the second human genome reference standard with sequencing data originating from the at least one preset simulated somatic mutation site in the sequencing data of the first human genome reference standard, thereby obtaining the simulated tumor reference standard sequencing data containing simulated somatic mutations, where the at least one preset simulated somatic mutation site is selected from the set of unique germline mutation sites.

According to the embodiments of this application, compared with a real tumor cell reference standard, the simulated tumor reference standard sequencing data constructed by the preceding method has the advantages of the number of mutations and a mutation frequency accurately controllable as required, completely clarified positive sites and negative sites, a reduced cost, and improved research efficiency; compared with directly modifying a base in sequencing data of a normal cell reference standard, the method has the advantage of a capability to retain real sequencing results.

In an implementation, the method for constructing the simulated tumor reference standard sequencing data includes the steps below.

In step (a), for the targeted region, the first germline mutation site set from the first human genome reference standard and the second germline mutation site set from the second human genome reference standard are acquired.

In this step, germline mutation site sets from different human genome reference standards are acquired to provide a basis for the subsequent construction of the simulated tumor reference standard sequencing data.

In this step, the first germline mutation site set and the second germline mutation site set used are separately derived from different human genome reference standards, that is, to construct the simulated tumor reference standard sequencing data, germline mutation sites need to be acquired from two different human genome standard samples. The reference standards are known reference samples used for scientific researches and subjected to quality control and generally contain specific genetic features, such as germline mutations. According to the embodiments of this application, the reference standards may have different germline mutations so that a mixture of two different reference standards can introduce a combination of simulated somatic mutations.

According to the embodiments of this application, germline mutation site datasets from different reference standards are used so that genomic features of a tumor reference standard can be more accurately simulated, the performance of a sequencing platform in detecting somatic mutations can be evaluated, and positives (mutation sites) and negative (non-mutation sites) can be clearly distinguished, thereby providing a means of comprehensively evaluating the performance of the sequencing technology and the bioinformatics algorithms and workflows.

According to an embodiment of this application, the reference standards may be from an authority such as an International genome sample bank (such as the Genome in a Bottle Consortium (GIAB)) or other biomedical research organizations that provide standard reference standards for genomics researches. In some examples of this application, the first human genome reference standard and the second human genome reference standard are each independently selected from at least one of HG001, HG002, HG003, HG004, or HG005, and the first human genome reference standard and the second human genome reference standard are different. The first germline mutation site set and the second germline mutation site set obtained from the reference standards are used for creating the simulated tumor reference standard, which can further help to evaluate and optimize the performance of analysis workflows, including the sensitivity, specificity, and accuracy of the analysis workflows.

According to an embodiment of this application, to construct the simulated tumor reference standard to obtain its sequencing data, all germline mutation sites of a genome are not necessarily used as a germline mutation site set, and only germline mutation sites associated with a region of interest (that is, the “targeted region”) may be used as a basis for constructing the simulated tumor reference standard sequencing data. The term “targeted region” used herein refers to a portion of a genome that receives particular attention during genome sequencing or another molecular analysis. According to an embodiment of this application, the targeted region is selected for in-depth researches due to the biological significance of the targeted region, a potential association of the targeted region with a disease, or the inclusion of specific functional genes in the targeted region. According to an embodiment of this application, the targeted region includes a specific contiguous DNA sequence pre-selected from the genome, such as one or more genes, a portion of a gene, a regulatory region, or another genetic marker. According to an embodiment of this application, the targeted region may include a gene associated with a specific biological function or disease, such as a susceptibility gene for a certain cancer. It is to be noted that the targeted region may be a small portion of the entire genome or include multiple dispersed regions, which depends on specific research requirements. In some examples of this application, the targeted region is determined based on specific high incidence regions of tumor somatic mutations. The germline mutation sites obtained based on the targeted region constitute a set of targeted germline mutation sites. The term “high incidence regions” used herein refers to specific genome fragments having relatively high mutation frequencies and associated with tumor occurrence, progression, or response to treatment in tumor somatic cells.

In some examples of this application, the germline mutations, such as SNVs or indels, are selected from high confidence intervals to encompass more mutations.

The germline mutations refer to mutations that occur in germ cells and that can be passed on to offspring. Unlike the somatic mutations, the germline mutations are present in all cells of an individual. Generally speaking, the germline mutations may affect a mechanism of tumorigenesis, including cell growth, differentiation, apoptosis, and other processes. According to an embodiment of the present application, in tumor sample sequencing, distinguishing the germline mutations from the somatic mutations is critical for reducing false positive results. The germline mutations are studied so that the accuracy of mutation detection can be improved. Additionally, the use of the germline mutations allows genomic researches on diseases without involving patient privacy and ethical issues. In an embodiment of this application, the “high confidence intervals” are selected from high confidence intervals of a cell reference standard given by the authority (such as the GIAB). The authority such as the GIAB gives the high confidence intervals and the germline mutations for the cell reference standard, while the germline mutations are not necessarily present in the high confidence intervals. If the germline mutations are within the high confidence intervals, both positive sites and negative sites have relatively high confidence. Positive sites and negative sites within non-high confidence intervals have relatively low confidence.

After the first germline mutation site set and the second germline mutation site set arc obtained, the method for constructing the simulated tumor reference standard sequencing data of the present application further includes step (b), that is, selecting, from the first germline mutation site set, the set of unique germline mutation sites relative to the second germline mutation site set.

According to the embodiments of this application, the first germline mutation site set and the second germline mutation site set are separately derived from two different human genome reference standards and both contain germline mutation information. The two sets are compared so that germline mutation sites that are present in the first germline mutation site set and that are not present in the second germline mutation site set can be identified, which are the unique germline mutation sites. The unique germline mutation sites are selected to construct the simulated tumor reference standard sequencing data used for the subsequent mutation detection performance evaluation. According to the embodiments of this application, the unique germline mutation sites are used so that a mutation detection capability of a sequencing platform, including sensitivity, specificity, and accuracy, can be evaluated.

In some examples of this application, after the set of unique germline mutation sites is obtained, the method may further include preprocessing the set of unique germline mutation sites. Thus, it can be ensured that the simulated tumor reference standard sequencing data is as accurate and reliable as possible, avoiding the influence of erroneous data on analysis results.

In some examples of this application, the preprocessing includes removing, from the set of unique germline mutation sites, the same low confidence germline mutation sites in the set of unique germline mutation sites and the second germline mutation site set. In an embodiment of this application, the second germline mutation site set may include the same germline mutation sites as the set of unique germline mutation sites. Due to relatively low confidence of these germline mutation sites, these germline mutation sites with relatively low confidence may fail to be identified when the unique germline mutation sites are determined. Therefore, to stimulate the specificity of the tumor reference standard, unique germline mutation sites that are also present in the second germline mutation site set need to be removed. Non-unique germline mutation sites are removed, and the preprocessing helps to purify the set of unique germline mutation sites so that the simulated data is closer to a research object. The unique germline mutation sites after the preprocessing more accurately reflect genetic features of a tumor sample, thereby improving the reliability of the simulated data in the subsequent analysis. In some examples of this application, the preprocessing includes removing at least one unique germline mutation site from the set of unique germline mutation sites based on position relationships between the unique germline mutation sites in the set of unique germline mutation sites.

In some examples of this application, the at least one unique germline mutation site is removed from the set of unique germline mutation sites based on the distances between the unique germline mutation sites in the set of unique germline mutation sites.

In some examples of this application, the preprocessing includes: determining the distances between the unique germline mutation sites in the set of unique germline mutation sites; and when a distance of the distances between the unique germline mutation sites is less than a predetermined distance threshold, removing the at least one unique germline mutation site from the set of unique germline mutation sites so that the distances between the remaining unique germline mutation sites are greater than or equal to the predetermined distance threshold.

In some examples of this application, the predetermined distance threshold is related to a sequencing read length of a sequencing platform for acquiring the sequencing data. If two unique germline mutation sites for simulation are too close to each other, one sequencing read generated through sequencing may contain more than one unique germline mutation site for simulation, resulting in mutual interference during simulation. Therefore, the distance threshold for the unique germline mutation sites for simulation is not less than 250 bp. Optionally, the distance threshold is 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, or 400 bp.

According to the embodiments of this application, the preprocessing takes into account the position relationships between the unique germline mutation sites, which can ensure the representativeness and distribution rationality of the unique germline mutation sites in the simulated data. If the distance between unique germline mutation sites is too small, one read generated during sequencing may contain multiple unique germline mutation sites, which may cause confusion and interference in analysis. Therefore, a unique germline mutation site too close to another unique germline mutation site needs to be removed. For the simulation accuracy, a minimum distance threshold is set to ensure that there is enough space between the unique germline mutation sites for simulation, thereby avoiding mutual interference. The threshold may be adjusted according to the read length of the sequencing platform and experimental designs. For the unique germline mutation sites whose distance does not exceed the predetermined threshold, the preprocessing causes at least one unique germline mutation site to be removed from the set of unique germline mutation sites so that the distances between the remaining unique germline mutation sites are equal to or greater than the predetermined distance threshold. This helps to reduce data complexity and maintain the representativeness of the simulated data.

According to the embodiments of this application, these optimization steps can improve the quality of the simulated data and ensure the reliability and effectiveness of the simulated tumor reference standard sequencing data in the subsequent analysis.

In an embodiment of this application, the method for constructing the simulated tumor reference standard sequencing data further includes step (c) of acquiring the sequencing data of the second human genome reference standard and the sequencing data of the first human genome reference standard, and for the at least one preset simulated somatic mutation site, in accordance with a predetermined replacement ratio, replacing sequencing data originating from the at least one preset simulated somatic mutation site in the sequencing data of the second human genome reference standard with sequencing data originating from the at least one preset simulated somatic mutation site in the sequencing data of the first human genome reference standard, thereby obtaining the simulated tumor reference standard sequencing data containing simulated somatic mutations, where the at least one preset simulated somatic mutation site is selected from the set of unique germline mutation sites.

Before this step, the first germline mutation site set and the second germline mutation site set are acquired from two different human genome reference standards, and the set of unique germline mutation sites is screened. Therefore, at least one specific unique germline mutation site may be selected as a target site in the simulated tumor reference standard sequencing data, which is predetermined for simulating somatic mutations in a tumor.

According to an embodiment of this application, for the preset simulated somatic mutation site, the sequencing data in the sequencing data of the second human genome reference standard and corresponding to the preset simulated somatic mutation site is replaced with the sequencing data in the sequencing data of the first human genome reference standard and corresponding to the preset simulated somatic mutation site according to a certain rule, where the preset simulated somatic mutation site is selected from the set of unique germline mutation sites. The purpose of this operation is to stimulate the somatic mutations in the tumor reference standard with the germline mutations. It is to be noted that the replacement operation is not performed randomly but is related to a mutation frequency of the preset simulated somatic mutation site. The mutation frequency refers to a probability or proportion of a specific mutation in the simulated tumor sample. The replaced data is derived from the sequencing data of the first human genome reference standard, which means that the replaced data is also obtained based on actual sequencing, ensuring the authenticity and reliability of the simulated data. Through the replacement operation, the simulated tumor reference standard sequencing data is finally generated. The introduction of the simulated data can basically solve the problems of a lack of reliable tumor reference standards and a difficulty in clarifying negative sites of a real tumor tissue reference standard. A comparison with other simulation methods is performed. Firstly, compared with a simulation method of mixing two normal cell reference standards at a sample level, the mixing at a sequencing data level can achieve flexible selections of the required somatic mutations and accurate control of frequencies of the somatic mutations, avoiding repeated sequencing for simulation schemes of different frequencies and greatly reducing a cost. Secondly, compared with a simulation method of modifying the base in the sequencing data of the normal cell reference standard, the mixing at the sequencing data level can always retain the real sequencing results. Additionally, the use of the simulated data allows genomic researches on diseases without involving patient privacy and ethical issues. Additionally, the simulated data can provide a controllable and standardized test environment for evaluating and optimizing the sequencing technology, analysis algorithms, and data processing workflows and training and testing machine learning models. The method can be widely used in the fields of biomedical research, drug development, clinical diagnosis, and personalized medicine and particularly valuable in tumor genomics research and precision medicine.

In the method, mutation sites and non-mutation sites are clarified to overcome the shortcoming of a failure to clarify negative sites in real tumor cell sequencing and achieve data standardization and controllability, thereby providing a consistent evaluation benchmark for different sequencing platforms and facilitating performance comparison. The method can reduce the false positive results and improve the detection accuracy and reliability and has flexibility and adjustability to adapt to different research requirements. According to the embodiments of this application, the mutation sites and the non-mutation sites are accurately identified in the method of this application so that the limitation that the negative sites cannot be determined in traditional sequencing methods is overcome. The clarification provides researchers with clear guidance and helps understand genetic variations in samples. Through standardization, the method ensures the consistency and comparability of the simulated data, which means that data generated by different laboratories or research teams can be compared and analyzed under the same criteria. Additionally, according to the embodiments of this application, mutation frequencies and mutation types in the simulated data can be controlled to simulate different characteristics of the tumor sample. The controllability provides great flexibility with which specific types of tumors are studied. The method can also provide a consistent evaluation benchmark for different sequencing platforms, making it possible to compare the performance of different sequencing platforms, which is of great significance for evaluating sequencing technologies and selecting the most appropriate sequencing technology. Through accurate site identification and a controllable simulation process, the method significantly reduces the false positive results and improves the detection accuracy and reliability. It is to be noted that by providing more accurate simulated data, the method of this application helps to promote the development of personalized medicine so that treatment plans can be customized according to specific genetic features of patients. The use of the simulated data allows the genomic researches on diseases without involving patient privacy and ethical issues, which is particularly important for researches requiring the processing of sensitive data.

For case of understanding, the method for constructing the simulated tumor reference standard sequencing data is explained in detail below through specific examples.

1) The first germline mutation site set from the first human genome reference standard HG002 and the second germline mutation site set from the second human genome reference standard HG001 are acquired.

HG001 represents a reference standard without somatic mutations, and HG002 represents a reference standard that is a source of the stimulated somatic mutations.

2) The set of unique germline mutation sites from the first germline mutation site set and relative to the second germline mutation site set is acquired.

An intersection between germline mutation sites of the two reference standards is computed based on a chromosome number (CHROM), position (POS), reference genome base (REF), and alternate base (ALT) of each germline mutation in the two reference standards, and germline mutation sites of HG002 that are not present in the intersection are the unique germline mutation sites of HG002 relative to HG001 (the dark part in FIG. 4).

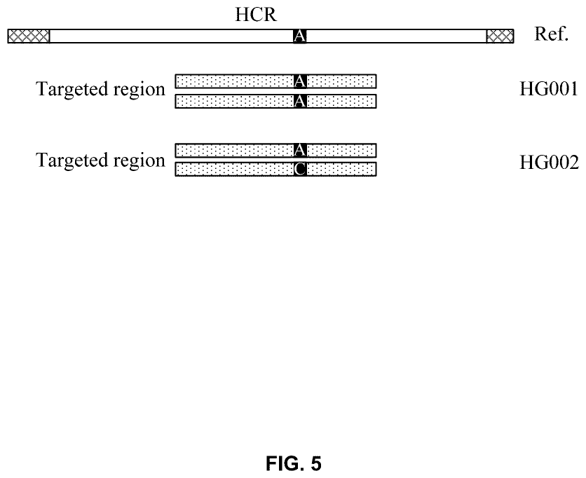

Specifically, in conjunction with genomes, as shown in FIG. 5, a heterozygous germline mutation at a site is present in HG002 and not present in HG001, and the site is one of the unique germline mutation sites of HG002 relative to HG001.

3) The predetermined replacement ratio for each site is determined based on the unique germline mutation sites.

For example, if a mutation frequency of 0.05 is to be obtained and a germline mutation at this site of HG002 is homozygous, the replacement ratio needs to be specified as 0.05; if the germline mutation at this site of HG002 is heterozygous, the replacement ratio is 0.05×2=0.1.

Further, the chromosome number, position, and replacement ratio of the site are written into a text document for simulation. In some examples of this application, the simulation is performed by software such as Posmix.

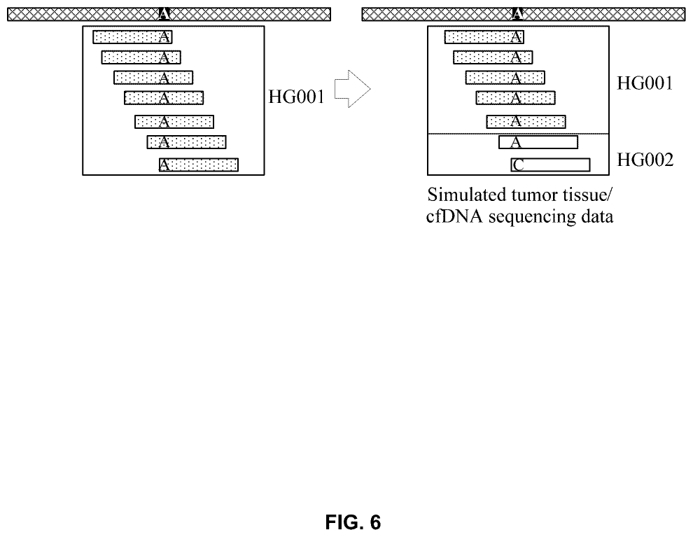

In a specific example of this application, post-alignment sequencing data (a bam file) without somatic mutations, post-alignment sequencing data (a bam file) of the source of the simulated somatic mutations, and the text document containing the chromosome number, position, and replacement ratio are input to Posmix software, and Posmix replaces reads at each specified site in the bam file of HG001 with reads of HG002 at the predetermined replacement ratio to form a new bam file containing the simulated somatic mutations, that is, obtain the sequencing data containing simulated somatic mutations (FIG. 6).

An embodiment 1 is provided below where the simulated tumor reference standard sequencing data containing simulated somatic mutations is constructed.

In this embodiment, with HG001 and HG002 as an example, a detailed construction process of the simulated tumor reference standard sequencing data containing simulated somatic mutations is demonstrated. HG001 represents the reference standard without somatic mutations, and HG002 represents the reference standard that is the source of the stimulated somatic mutations. Specific steps are described below.

(1a) The germline mutation sites of HG001 and HG002 in the targeted region are acquired.

By use of the bedtools software, bam files of the two reference standards and bed files of the targeted region are separately introduced into a Linux system to acquire the germline mutation sites of HG001 and HG002 in the targeted region.

(2a) An intersection between the germline mutation sites of HG001 and HG002 in the targeted region is computed, and the unique germline mutation sites of HG002 relative to HG001 are obtained.

In R, the intersection between high confidence germline mutations of two cell reference standards in the targeted region is computed, and two sites with the same chromosome number (CHROM) and the same position (POS) in the two reference standards are considered as the same sites. Thus, the finally obtained unique germline mutation sites of HG002 are germline mutation sites that are not present in HG001.

(3a) The set of unique germline mutation sites satisfying the predetermined distance threshold is screened.

The unique germline mutation sites are sorted with CHROM as a primary keyword and POS as a secondary keyword (if R is used, a default sorting manner is used without a need to write codes for sorting). Then, the distances between each unique germline mutation site and two unique germline mutation sites adjacent to this unique germline mutation site are computed. The distance between the first unique germline mutation site on a certain chromosome and the previous unique germline mutation site is recorded as positive infinity; and the distance between the last unique germline mutation site on a certain chromosome and the next unique germline mutation site is recorded as negative infinity. The unique germline mutation sites satisfying the distance threshold not less than 250 bp are selected.

(4a) A germline mutation site actually contained in HG001 is removed from the unique germline mutation sites of HG002.

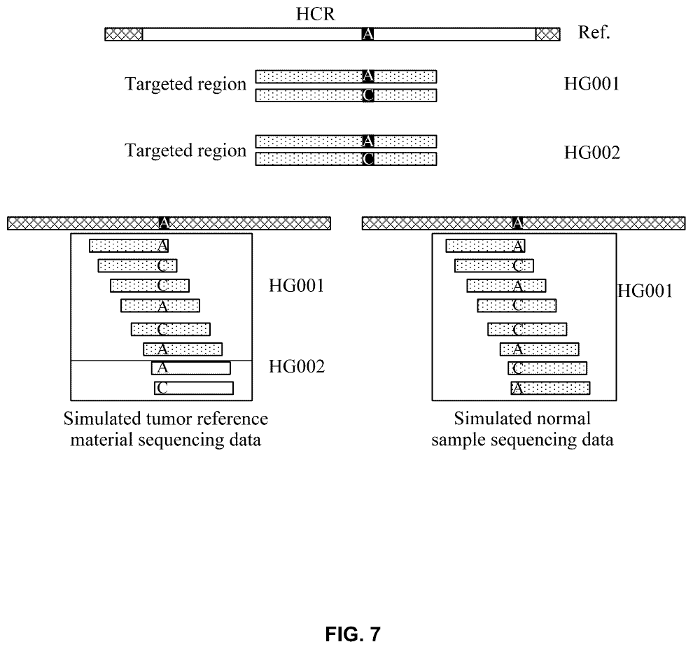

Theoretically, after step 2, HG001 should not contain any of the obtained unique germline mutation sites of HG002. However, as can be known from the actual sequencing data of HG001, HG001 still contains some germline mutation sites the same as some of the unique germline mutation sites of HG002, and these germline mutation sites are not reported by the GIAB due to low confidence and need to be removed. As shown in FIG. 7, HG001 has the same heterozygous germline mutation as HG002 at a certain site, and so a somatic mutation cannot be simulated at this site.

(5a) The simulated mutation frequency is determined to be 5%, and the replacement ratio for each site is generated with consideration of the homozygous/heterozygous germline mutation of HG002.

In step 4, all correct unique germline mutation sites that can be used for simulation are obtained. Considering that a computational amount may be too large, part of the unique germline mutation sites may be extracted. However, no extraction is performed and all the unique germline mutation sites are used for simulation in this embodiment. The replacement ratio needs to be computed according to the mutation frequency and the homozygous/heterozygous germline mutation of HG002 at this site, and the text document introduced into Posmix is generated.

(6a) The simulated tumor reference standard sequencing data containing simulated somatic mutations is generated.

A volume of data to be stimulated is determined, and reads corresponding to this volume of data are randomly extracted from the bam file of HG001 (the ratio of the volume of data to an existing volume of data needs to be computed, and a seed value used is determined, where a seed value of 100 is used and the ratio is 0.1 in this embodiment). Then, the Posmix software is started, and three parameters are introduced for simulation to generate the simulated tumor reference standard sequencing data containing simulated somatic mutations.

It is to be understood that the method for constructing the simulated tumor reference standard sequencing data according to the embodiments of this application is an independent method for acquiring tumor reference standard sequencing data and can be applied to any solution requiring the tumor reference standard sequencing data. For example, the gene mutation detection method in the embodiments of this application adopts the method for constructing the simulated tumor reference standard sequencing data to acquire the required training sequencing data, but the method for constructing the simulated tumor reference standard sequencing data is not only used for this purpose.

A use of the simulated tumor reference standard sequencing data for evaluating efficiency of tumor sample detection of a sequencing platform is described below in conjunction with embodiments.

In another aspect of this application, this application provides the use of the simulated tumor reference standard sequencing data for evaluating the efficiency of tumor sample detection of the sequencing platform, where the simulated tumor reference standard sequencing data is obtained by the preceding method for constructing the simulated tumor reference standard sequencing data.

An embodiment 2 is provided below where an effect of the simulated tumor reference standard sequencing data containing simulated somatic mutations is verified.

In this embodiment, mutation frequency gradients (0.5%, 0.75%, 1%, 1.25%, and 1.5%) are simulated by using sequencing data of HG001 and HG002 from three sequencing platforms FASTASeq 300, GenoLab M, and NovaSeq 6000, three somatic mutation detection software Strelka2, Mutect2, and VarScan are used, and recalls and precisions under different sequencing platforms, mutation frequencies, and detection software are demonstrated, so as to evaluate the performance of different detection software under different sequencing platforms and the mutation frequency gradients and horizontally compare the sequencing platforms in somatic mutation detection performance.

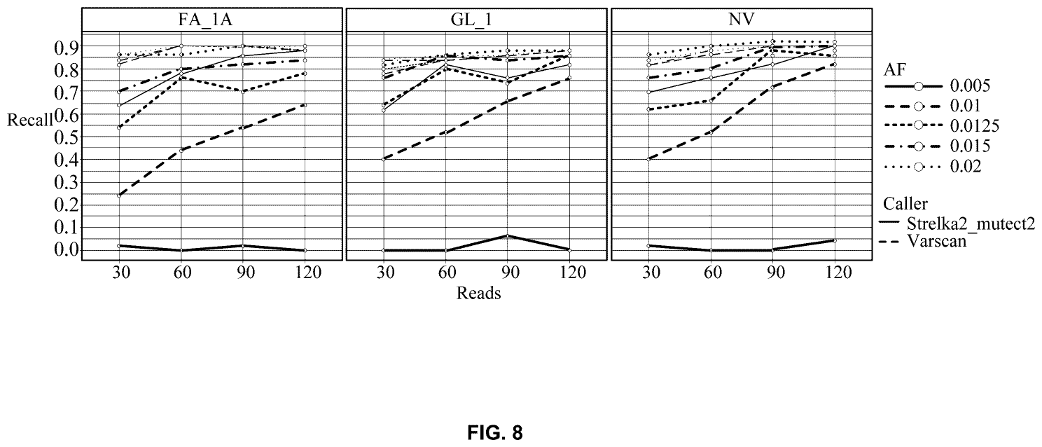

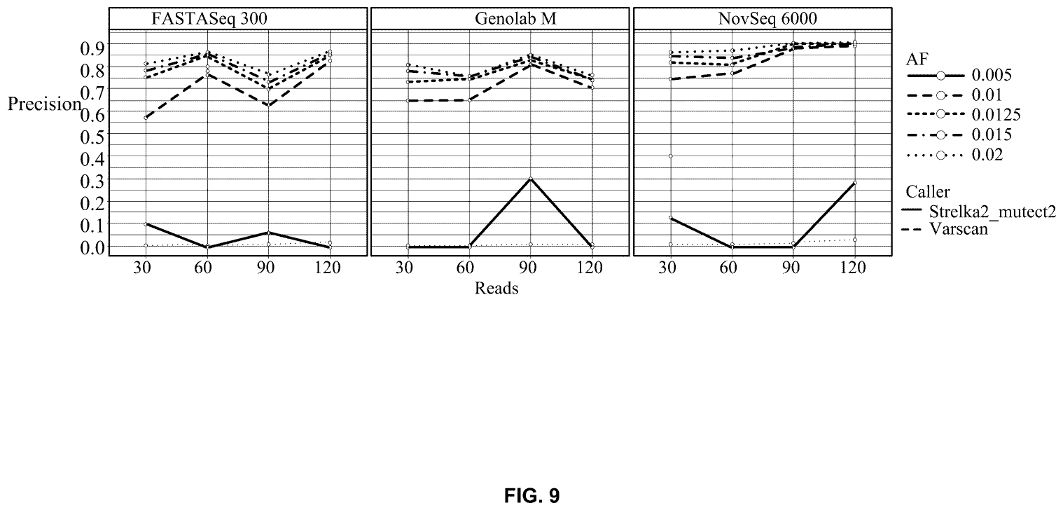

Based on statistical inference methods (such as a somatic likelihood model of Mutect2), Mutect2 and Strelka2 compute multiple statistical quantities for each candidate mutation and set thresholds for filtering to distinguish true mutations from sequencing errors. Mutect2 and Strelka2 are characterized by high precisions and low recalls for low-frequency mutations. Therefore, FIGS. 8 and 9 show a union of Mutect2 and Strelka2 (Strelka2_Mutect2). In contrast, VarScan cannot directly distinguish true mutations from sequencing errors and tends to exhibit high recalls and generally low precisions.

With increases in the sequencing depth and/or the mutation frequency, the recall generally increases until the recall reaches a plateau. However, when Strelka2 or Mutect2 is used, if the mutation frequency is too low (for example, 0.5%), the recall cannot be increased even if the sequencing depth is increased. The precision is strongly correlated to the detection software and weakly correlated to the sequencing platform and the sequencing depth. Strelka2 and Mutect2 generally exhibit high precisions, while VarScan exhibits low precisions.

The simulation method has stable performance and can accurately and reliably identify positive sites and negative sites, reduce a cost, and improve research efficiency.

In some examples of this application, the simulated tumor reference standard sequencing data is stored in a server.

In some examples of this application, the tumor sample detection is performed using the sequencing platform. The sequencing platform is as described above and is not repeated here.

In some examples of this application, evaluating the efficiency of tumor sample detection includes evaluating a recall and a precision. Evaluating the recall and the precision is of great significance for tumor sample detection. The recall measures a capability of the detection method to identify all actually occurring mutations and reflects the sensitivity of the detection method, ensuring that as few mutations as possible fail to be detected. The precision measures an overall capability of the detection method to accurately identify mutations and reflects the reliability and accuracy of the detection result. The two indicators are evaluated so that researchers can fully understand the performance of the detection method, optimize a detection workflow, select an optimal sequencing platform, and ensure that clinical decisions are based on high-quality data, thereby improving the effects of personalized treatments.

Based on the use of the simulated tumor reference standard obtained above for evaluating the efficiency of tumor sample detection of the sequencing platform, the performance of the sequencing platform can be comprehensively evaluated, the credibility and reliability of analysis results can be ensured, clinical decision-making can be guided, and a cost and a time can be saved.

### Method for Implementing an Analysis Workflow

In another aspect of this application, this application provides the method for implementing an analysis workflow. The method includes: inputting simulated tumor reference standard sequencing data into the analysis workflow to obtain analysis results of the analysis workflow, where the simulated tumor reference standard sequencing data is constructed using preset simulated somatic mutation sites by the preceding method for constructing the simulated tumor reference standard sequencing data; and comparing the analysis results with the preset simulated somatic mutation sites for constructing the simulated tumor reference standard sequencing data to determine a recall and a precision of the analysis workflow.

The simulated tumor reference standard sequencing data constructed using the preset simulated somatic mutation sites is input into the analysis workflow, and the analysis results are compared with these preset simulated somatic mutation sites so that the recall and the precision of the analysis workflow can be accurately computed. This process enables researchers to fully understand and evaluate the performance of the analysis workflow in mutation detection. The accurate evaluation of the recall and the precision can help optimize and improve the detection method, select an optimal sequencing platform, and ensure the reliability and accuracy of the analysis results, thereby improving the effects of personalized treatments. Additionally, the method provides a standardized evaluation means, making performance comparisons between different analysis workflows and different sequencing platforms more consistent and repeatable.

The training sequencing data of the training nucleic acid sample refers to nucleic acid sequence data of the training nucleic acid sample. The nucleic acid sequence data of the training nucleic acid sample may be all sequence data of the training nucleic acid sample, partial sequence data of the training nucleic acid sample including gene variant sites, or even types of bases at a gene mutation site. The types of bases at the gene mutation site include a type of base before the mutation and a type of base after the mutation.

In an embodiment of this application, the first mutation detection module performs feature extraction on the training sequencing data of the training nucleic acid sample and acquires the training mutation site based on extracted features. That is, the training mutation site is determined by first training feature data obtained from mutation detection of the first mutation detection module on the sequencing data of the training nucleic acid sample. Each training nucleic acid sample may contain one training mutation site or two or more training mutation sites. The first mutation detection module is as described above, and for the acquisition of the training mutation site of the training nucleic acid sample by the first mutation detection module, reference is made to the content of “acquiring a suspected mutation site of a nucleic acid sample under test”. To save space, the details are not repeated here. Further, secondary confirmation may be performed on the “suspected mutation site” obtained by the preceding method to confirm whether the suspected mutation site is a true or false positive.

(2) Second training feature data and third training feature data of the training mutation site are input into an initial mutation detection model to be trained so that the output of a predicted mutation detection result of the training mutation site is obtained.

In step (2), the second training feature data is feature data obtained from feature extraction of the second mutation detection module on each training mutation site, and the third training feature data is acquired as follows: the sequencing data processing module processes the training sequencing data to obtain total processed training sequencing data and screens processed sequencing data of each training mutation site from the total processed training sequencing data to obtain the third training feature data. The second mutation detection module and the sequencing data processing module are not described in detail here.

In some embodiments, the second training feature data includes the preceding second mutation feature data, and the third training feature data includes the preceding third mutation feature data, which are not described in detail here.

For each training mutation site, the second training feature data acquired by the second mutation detection module and the third training feature data acquired by the sequencing data processing module are acquired and inputted into the initial mutation detection model to be trained, and training is performed so that the output of the predicted mutation detection result of the training mutation site is obtained. In an embodiment of this application, the initial mutation detection model is an untrained initial model of the target mutation detection model.

(3) The initial mutation detection model is trained based on the predicted mutation detection result and a standard mutation detection result corresponding to the training mutation site so that the target mutation detection model trained is obtained.

In this step, the initial mutation detection model is trained based on the predicted mutation detection result obtained in step (2) and the standard mutation detection result corresponding to the training mutation site in step (1) so that the target mutation detection model trained is obtained. It is to be understood that for the same training mutation site, a one-to-one correspondence relationship exists between the predicted mutation detection result obtained in step (2) and the standard mutation detection result corresponding to the training mutation site in step (1).

In an embodiment of this application, a model algorithm of the target mutation detection model is not strictly limited. In some embodiments, the model algorithm of the target mutation detection model is a gradient boosting model. For example, the model algorithm of the target mutation detection model includes, but is not limited to, a logical classification model, a decision tree, a support vector machine, random forests, an Adaptive Boosting (AdaBoost) model, an eXtreme Gradient Boosting (XGBoost) model, or a deep confidence network, which may be customized according to actual requirements.

Specifically, that the initial mutation detection model is trained based on the predicted mutation detection result and the standard mutation detection result corresponding to the training mutation site so that the target mutation detection model trained is obtained includes: determining a loss function according to the predicted mutation detection result and the standard mutation detection result corresponding to the training mutation site, adjusting a model parameter of the initial mutation detection model according to the loss function until a preset ending condition is satisfied, and using the initial mutation detection model in a current iteration process as the target mutation detection model trained.

For example, a type of the loss function includes, but is not limited to, a square loss function, a log loss function, an exponential loss function, a mean squared error loss function, a logistic regression loss function, a Huber loss function, a cross-entropy loss function, or a Kullback-Leibler divergence loss function. The type of the loss function is not limited here and may be customized according to actual requirements.

Based on the preceding embodiments, optionally, before the initial mutation detection model in the current iteration process is used as the target mutation detection model verified, the method further includes: acquiring a verification mutation site in verification sequencing data of a verification nucleic acid sample; acquiring second verification feature data and third verification feature data of the verification mutation site extracted by the second mutation detection module and the sequencing data processing module, respectively, and inputting the second verification feature data and the third verification feature data into the initial mutation detection model in the current iteration process to obtain the output of a verification mutation detection result of the verification mutation site; and determining model performance of the initial mutation detection model according to the verification mutation detection result, and in the case where the model performance satisfies a preset performance condition, using the initial mutation detection model in the current iteration process as the target mutation detection model verified.

For example, the model performance includes, but is not limited to, at least one of the recall, the precision, an F1 score, an accuracy, or the area under a receiver operating characteristic (ROC) curve. The model performance is not limited here and may be customized according to actual requirements.

Based on the preceding embodiments, the method further includes: in the case the model performance does not satisfy the preset performance condition, adjusting a hyperparameter of the initial mutation detection model, and returning to the step of acquiring the training mutation site in the training sequencing data of the training nucleic acid sample.

Based on the preceding embodiments, optionally, an architecture of the target mutation detection model is a classification model; and before the second mutation feature data and the third mutation feature data are input into the pre-trained target mutation detection model and the mutation detection result of each suspected mutation site is output, the method further includes: screening the second mutation feature data according to feature weights corresponding to at least two second mutation features in the second mutation feature data; and screening the third mutation feature data according to feature weights corresponding to at least two third mutation features in the third mutation feature data. Each feature weight is determined by the target mutation detection model during the last iterative training.

In some embodiments, screening the second mutation feature data according to the feature weights corresponding to the at least two second mutation features in the second mutation feature data includes: screening second mutation features whose feature weights are greater than a first weight threshold as the screened second mutation features. For example, the first weight threshold may be 0.001, and the first weight threshold is not limited here and may be customized according to actual situations.

In some embodiments, screening the second mutation feature data according to the feature weights corresponding to the at least two second mutation features in the second mutation feature data includes: sorting the at least two second mutation features according to the feature weights corresponding to the at least two second mutation features, and determining the screened second mutation features according to a sorting result and a first selection number or a first selection proportion. For example, the first selection number may be 5, and the first selection proportion may be 50%. The first selection number or the first selection proportion is not limited here and may be customized according to actual requirements.

In some embodiments, screening the third mutation feature data according to the feature weights corresponding to the at least two third mutation features in the third mutation feature data includes: screening third mutation features whose feature weights are greater than a second weight threshold as the screened third mutation features. For example, the second weight threshold may be 0.0001, and the second weight threshold is not limited here and may be customized according to actual situations.

In some embodiments, screening the third mutation feature data according to the feature weights corresponding to the at least two third mutation features in the third mutation feature data includes: sorting the at least two third mutation features according to the feature weights corresponding to the at least two third mutation features, and determining the screened third mutation features according to a sorting result and a second selection number or a second selection proportion. For example, the second selection number may be 10, and the second selection proportion may be 60%. The second selection number or the second selection proportion is not limited here and may be customized according to actual requirements.

Such an arrangement has the following advantages: a data volume of mutation features input into the target mutation detection model can be reduced, and a computational amount of the target mutation detection model can be increased, thereby improving the efficiency of gene mutation detection.

According to the technical solutions of this embodiment, at least two pre-trained reference mutation detection models are acquired, and the first mutation detection module and the second mutation detection module are determined from the at least two reference mutation detection models according to a recall and a precision corresponding to each reference mutation detection model, thereby solving the problem of manual screening of the first mutation detection module and the second mutation detection module and improving the screening efficiency of the first mutation detection module and the second mutation detection module while ensuring that the first mutation detection module and the second mutation detection module satisfy performance constraint conditions.

A description is given below in conjunction with specific examples.

(1A) Simulated somatic mutations are analyzed by using three reference standards from two sequencing platforms.

The three reference standards, HG001, HG002, and HG003, are used so that six site sets can be obtained, each set having approximately 300 sites. Capture sequencing is performed using a tumor panel from iGeneTech, and each reference standard from each sequencing platform has three replicates of sequencing data, where each replicate of sequencing data includes reads of greater than 160 M. During simulation, five volumes of tumor data are used, which are set to 50 M, 70 M, 100 M, 130 M, and 150 M.

(2A) Mutation detection results of VarScan software are used for running Mutect2 software.

(3A) Second training feature data extracted by the Mutect2 software is read, and third training feature data is read from a readcount file, so as to form structured data.

(4A) A true mutation site and false positive mutation sites are labeled.

| | | | | | | | | | | | | | la- | |
|---|---|---|---|---|---|---|---|---|---|---|---|---|---|---|
| Training | DP | ECNT | GERMQ | MBQ1 | MBQ2 | MFRL1 | MFRL2 | MMQ1 | MMQ2 | NALOD | NLOD | TLOD | bel | amq |
| 10:104352334:C:T | 1579 | 1 | 93 | 20 | 20 | 181 | 180 | 60 | 60 | 1.83 | 19.86 | 1.1000 | 0 | 60 |
| 10:104352446:G:A | 2393 | 1 | 93 | 20 | 20 | 168 | 162 | 60 | 60 | 2.00 | 29.45 | 3.0300 | 0 | 60 |
| 10:104377101:G:T | 2194 | 2 | 93 | 20 | 20 | 170 | 161 | 60 | 60 | 2.01 | 30.08 | 2.9900 | 0 | 60 |
| 10:104590740:G:T | 1733 | 1 | 93 | 20 | 20 | 182 | 131 | 60 | 60 | 1.93 | 25.17 | 1.0100 | 0 | 60 |
| 10:104592744:G:T | 1002 | 2 | 93 | 20 | 28 | 194 | 208 | 60 | 60 | 1.83 | 19.22 | 2.6400 | 0 | 60 |
| 10:104592816:C:A | 1986 | 2 | 93 | 20 | 20 | 174 | 118 | 60 | 60 | 2.05 | 32.87 | 1.0400 | 0 | 60 |
| 10:104594665:C:A | 1787 | 2 | 93 | 20 | 20 | 173 | 136 | 60 | 60 | 1.93 | 25.19 | 0.9740 | 0 | 60 |
| 10:104597114:C:T | 1413 | 1 | 93 | 20 | 20 | 190 | 167 | 60 | 60 | 1.84 | 20.12 | 1.2400 | 0 | 60 |
| 10:104849438:C:T | 2176 | 2 | 93 | 20 | 20 | 171 | 143 | 60 | 60 | 1.76 | 33.31 | 0.2660 | 0 | 60 |
| 10:104849648:C:A | 1670 | 1 | 93 | 20 | 20 | 171 | 148 | 60 | 60 | 1.86 | 21.36 | 1.0600 | 0 | 60 |
| 10:104866451:G:T | 3211 | 1 | 93 | 20 | 5 | 176 | 190 | 60 | 60 | 1.88 | 44.33 | 0.0150 | 0 | 60 |
| 10:104934688:G:T | 2242 | 2 | 93 | 20 | 20 | 168 | 241 | 60 | 60 | 2.01 | 30.00 | 2.1700 | 0 | 60 |
| 10:104934709:T:C | 1960 | 2 | 93 | 20 | 20 | 173 | 143 | 60 | 60 | 1.97 | 27.00 | 41.3500 | 1 | 60 |
| 10:111967617:G:T | 770 | 2 | 93 | 20 | 20 | 185 | 254 | 60 | 60 | 1.56 | 10.52 | 1.9300 | 0 | 60 |
| 10:111967644:G:T | 914 | 2 | 93 | 20 | 20 | 184 | 169 | 60 | 60 | 1.67 | 13.52 | −0.2366 | 0 | 60 |
| 10:111967816:G:T | 1401 | 2 | 93 | 20 | 20 | 169 | 123 | 60 | 60 | 1.81 | 18.24 | 3.8900 | 0 | 60 |
| 10:112044711:C:T | 1894 | 2 | 93 | 20 | 17 | 176 | 166 | 60 | 60 | 1.83 | 19.72 | 0.8520 | 0 | 60 |
| 10:112044736:C:A | 1491 | 2 | 93 | 20 | 28 | 182 | 155 | 60 | 60 | 1.74 | 16.21 | 1.9300 | 0 | 60 |
| 10:112724158:A:G | 3034 | 1 | 93 | 20 | 20 | 178 | 182 | 60 | 60 | 2.11 | 38.22 | 3.7100 | 0 | 60 |
| 10:112764549:G:T | 1759 | 1 | 93 | 20 | 20 | 195 | 123 | 60 | 60 | 1.98 | 27.90 | 0.9750 | 0 | 60 |

For example, with a training mutation site “10:104352334:C:T” in Table 1 as an example, “10” represents a chromosome number, “104352334” represents a gene mutation position, “C” represents a base at the gene mutation position of the training mutation site, and “T” represents a base at the gene mutation position of a reference genome. MBQ1 and MBQ2 represent a Median Base Quality of an alternate allele and a Median Base Quality of a reference allele, respectively; MFRL1 and MFRL2 represent a Median Fragment Length of the alternate allele and a Median Fragment Length of the reference allele, respectively; MMQ1 and MMQ2 represent a Median Mapping Quality of the alternate allele and a Median Mapping Quality of the reference allele, respectively; and label represents labeling results of the true mutation site and the false positive mutation sites, for example, “1” indicates that the training mutation site is the true mutation site, and “0” indicates that the training mutation site is a false positive mutation site.

(5A) An XGBoost model is trained, verified, and tested.

In an embodiment of this application, the mutation detection results of the VarScan software are used for running the Mutect2 software to re-compute feature values (that is, second mutation feature data) of each training mutation site output from the VarScan software, and after the true mutation site and the false positive mutation sites are separately labelled, the XGBoost model is trained by using the feature values in conjunction with third mutation feature data (that is, features such as an average base quality value and an average mapping quality value) extracted from the readcount file so that the XGBoost model can reclassify the mutation detection results of the VarScan software to obtain the true mutation site.

The Mutect2 software constructs a series of feature values by which the true mutation site can be accurately distinguished from the false positive mutation sites for a mutation frequency >1.5%. Although the Mutect2 software cannot obtain a high recall for a mutation frequency <1.5%, a powerful learning model for distinguishing true mutation sites from false positive mutation sites can be trained by a machine learning method using the feature values in conjunction with the third mutation feature data in the readcount file.

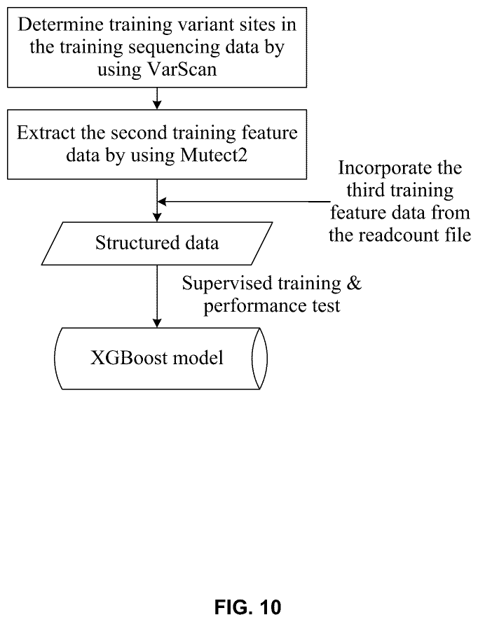

FIG. 10 is a flowchart of a method for training an XGBoost model according to an embodiment of this invention. In FIG. 10, for example, the first mutation detection module is implemented using the Varscan software, and the second mutation detection module is implemented using the Mutect2 software. Specifically, the Varscan software determines a training mutation site in training sequencing data. The Mutect2 software extracts second training feature data of the training mutation site. Structured data is constructed according to the second training feature data and third training feature data in a readcount file of a sequencing data processing module. Supervised training is performed on the XGBoost model according to the structured data. After the supervised training is completed, performance testing is performed on an XGBoost model in a current iteration process according to verification sequencing data of a verification nucleic acid sample. After passing the performance testing, the XGBoost model in the current iteration process is used as a trained XGBoost model.

TABLE 2
shows model parameter data of the XGBoost model
according to an embodiment of this invention.

| Feature | Gain | Cover | Frequency |
|---|---|---|---|
| TLOD | 0.784873803268618 | 0.31711059012154 | 0.129680328528867 |
| DP | 0.06038765225713 | 0.148886033883581 | 0.118367018278444 |
| avg_sum_mismatch_{—} | 0.0306830985752937 | 0.139568722896268 | 0.108825187213141 |
| qualities | | | |
| avg_mapping_quality | 0.0262767435549719 | 0.0225388591485286 | 0.0334165391738465 |
| NALOD | 0.022590189492459 | 0.0567874075463778 | 0.0826556083420565 |
| avg_pos_as_fraction | 0.0113849223690149 | 0.0376182868444917 | 0.0687253402045253 |
| NLOD | 0.0100662549462865 | 0.0330585319296923 | 0.08728561075771 |
| MBQ_ALT | 0.00999495741651965 | 0.0810024386724121 | 0.0324905386907158 |
| ECNT | 0.00931290556558733 | 0.0177370826318517 | 0.0411868910540301 |
| MFRL_ALT | 0.00872215089080331 | 0.0333552073856335 | 0.074844995571302 |
| avg_num_mismatches_{—} | 0.0069506581111646 | 0.0164293798623841 | 0.0241967952331106 |
| as_fraction | | | |
| avg_basequality | 0.00608960310639945 | 0.0265618552019328 | 0.0485546340285047 |
| MFRL_REF | 0.00595906018978996 | 0.0300614495238704 | 0.0739189950881714 |
| MPOS | 0.00412038860156649 | 0.0254523510846998 | 0.0555197680972703 |
| MMQ_REF | 0.00162211954061967 | 0.00764738230184571 | 0.00797165633303809 |
| MBQ_REF | 0.000596965207689395 | 0.00316169215510078 | 0.00789113455189629 |
| MMQ_ALT | 0.000368526906086068 | 0.00302272880978946 | 0.00446895885336984 |

“Feature” represents an input mutation feature, “Gain” represents a feature weight, “Cover” represents coverage, and “Frequency” represents a frequency.

In the above specific examples, the trained XGBoost model is used to filter somatic mutation detection results output from the VarScan software, where 0.5 is tentatively used as a cutoff for predicted values. Three illustrative embodiments are given below.

1) With HG004 as a reference standard that is a source of somatic mutations and HG005 as a reference standard without somatic mutations, simulated somatic mutation detection results from sequencing platforms GenoLab M and FASTASeq 300 are filtered. Finally, the XGBoost model has a recall of 0.93 and a precision of 0.88.

2 With HG002 as a reference standard that is a source of somatic mutations and HG001 as a reference standard without somatic mutations, simulated somatic mutation detection results from sequencing platforms NovaSeq 6000 and Element AVITI are filtered. Finally, the XGBoost model has a recall of 0.95 and a precision of 0.95.

The following describes a gene mutation detection apparatus according to an embodiment of this invention. The gene mutation detection apparatus of this embodiment and the gene mutation detection method of any previous embodiment belong to the same inventive concept. For details not described in the embodiment of the gene mutation detection apparatus, reference may be made to embodiments of the gene mutation detection method.

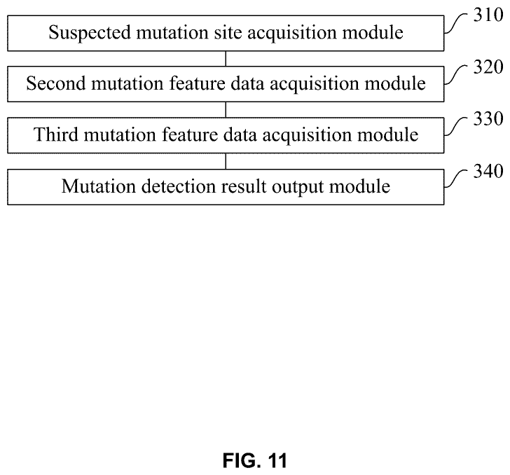

FIG. 11 is a diagram illustrating the structure of a gene mutation detection apparatus according to an embodiment of this invention. As shown in FIG. 11, the apparatus includes a suspected mutation site acquisition module 310, a second mutation feature data acquisition module 320, a third mutation feature data acquisition module 330, and a mutation detection result output module 330.

The suspected mutation site acquisition module 310 is configured to acquire a suspected mutation site of a nucleic acid sample under test.

The second mutation feature data acquisition module 320 is configured to perform feature extraction through a second mutation detection module on each suspected mutation site to obtain second mutation feature data.

The third mutation feature data acquisition module 330 is configured to process the sequencing data through a sequencing data processing module to obtain overall processed sequencing data and screen processed sequencing data of each suspected mutation site from the overall processed sequencing data to obtain third mutation feature data.

The mutation detection result output module 340 is configured to input the second mutation feature data and the third mutation feature data into a pre-trained target mutation detection model and output a mutation detection result of each suspected mutation site.

Specifically, the nucleic acid sample under test represents a sample containing nucleic acid molecules or nucleic acid mixtures and used for mutation detection. The nucleic acid mixture includes two or more types of nucleic acid molecules having different sequences. In embodiments of this invention, the term “nucleic acid molecule” refers to a polymer of nucleotides of any length. The nucleic acid molecule may be a short nucleic acid fragment less than or equal to 500 bp, for example, 20 bp, 50 bp, 80 bp, 100 bp, 120 bp, 150 bp, 180 bp, 200 bp, 220 bp, 250 bp, 280 bp, 300 bp, 320 bp, 350 bp, 380 bp, 400 bp, 420 bp, 450 bp, 480 bp, or 500 bp, or may be a long nucleic acid fragment longer than 500 bp. In embodiments of this invention, the “nucleic acid molecule” includes a nucleic acid molecule composed of ribonucleotides or a nucleic acid molecule composed of deoxyribonucleotides. Moreover, the nucleic acid sample under test in embodiments of this application may be an amplified nucleic acid sample. That is, for each nucleic acid molecule sequence in the nucleic acid sample under test, there are multiple copies of that nucleic acid molecule.

By way of example, the sample sources include, but are not limited to, blood, urine, plasma, cell samples, ex vivo tissue fluid, or tumor tissue. Here the sample sources of the nucleic acid sample under test are not limited. In an embodiment, the sample is a nucleic acid sample under test derived from humans. That is, the gene mutation detection method of embodiments of this application is a gene mutation detection method for human samples. It is to be understood that this method is also applicable to gene mutation detection of nucleic acid sample under tests derived from other biological species. In the following description, human-derived nucleic acid sample under tests are used as examples but are not intended to limit the species source of the nucleic acid sample under tests.

In some embodiments, the nucleic acid sample under test has a mutation frequency less than or equal to 1.5%. Specifically, the gene mutation frequency refers to, for a sample, at a particular genomic locus, the ratio of the sequencing depth (Read Depth (DP)) of a specific mutation to the total sequencing depth (Read Depth (DP)). For example, in the reference genome, if the total number of bases at genomic locus P is 100 (that is, the sequencing depth (Read Depth (DP)) is 100) and the base is adenine A (meaning that without mutation, the base detected at the genomic locus P is theoretically adenine A). In the gene sequencing data of the nucleic acid sample under test, the genomic locus P has a cytosine (C) mutation with 5 bases (that is, the sequencing depth (Read Depth (DP)) is 5), in other words, five adenine (A) bases at genomic locus P are mutated to cytosine (C), then the gene mutation frequency in the nucleic acid sample under test is 5%.

Traditional gene mutation detection software performs poorly on nucleic acid samples with mutation frequencies less than or equal to 1.5%, failing to ensure both high precision and high recall. The target mutation detection model in this embodiment is especially suitable for mutation detection of nucleic acid samples with mutation frequencies less than or equal to 1.5%, capable of balancing high precision and high recall of detection results.

In some embodiments, the nucleic acid sample under test is a biological sample with somatic mutations or a biological sample with germline mutations. Somatic mutations refer to mutations arising in somatic cells during individual development due to mutagenic factors, which generally are not inherited by offspring. Germline mutations refer to mutations carried by germ cells during sexual reproduction, which can be inherited by offspring.

Specifically, the sequencing data of the nucleic acid sample under test represents nucleic acid sequence obtained by sequencing the sample using sequencing technology. For example, sequencing technologies include, but are not limited to, Sanger sequencing, high-throughput sequencing, and single-molecule sequencing. Sequencing technologies are not limited here and can be configured as required. Sequencing may be single-end or paired-end. Sequencing can be performed on sequencing platforms including, but not limited to, Illumina's Hiseq/Miseq/Nextseq/Novaseq platform, Thermo Fisher/Life Technologies' Ion Torrent platform, BGI's BGISEQ and MGISEQ platforms, single-molecule sequencing platforms, and GeneMind's GenoLab M/GenoCare 1600/FASTASeq 300/SURFSeq 5000 platform.

In embodiments of this application, the mutation site refers to a genomic locus in the nucleic acid sample where a base mutation causes the sequence to differ from the reference genome. It is to be understood that in embodiments of this application, mutation sites belong to the category of variant sites, with the ratio of mutation sites to variant sites ranging between 0 and 1.

In embodiments of this application, the suspected mutation sites are determined by first mutation feature data obtained from mutation detection of a first mutation detection module on sequencing data of the nucleic acid sample under test. Analyzing the sequencing data of the nucleic acid sample under test with the first mutation detection module can extract mutation features inherently contained in the sequencing data and/or mutation features obtained after further data processing of the sequencing data. In embodiments of this application, the mutation feature data obtained by the first mutation detection module is referred to as first mutation feature data. That is, in embodiments of this application, the suspected mutation sites are determined by first mutation feature data obtained from mutation detection of a first mutation detection module on sequencing data of the nucleic acid sample under test.

In embodiments of this application, when the first mutation detection module for mutation detection of the nucleic acid sample under test identifies gene mutation sites, that is, performs mutation detection, the recall is greater than or equal to a preset recall, that is, the recall in identifying gene mutation sites is greater than or equal to a preset recall, ensuring that the probability of identifying true mutation sites satisfies a preset probability requirement. Thus, the suspected mutation sites obtained by the first mutation detection module are then passed to the second mutation feature data acquisition module 320, where a second mutation detection module satisfying a preset precision requirement further analyzes them. The second mutation feature data extracted by the second mutation detection module is used as one of the input features of the target mutation detection model. This allows the target mutation detection model in the mutation detection result output module 340 to output mutation detection results balancing high recall and high precision.

In some embodiments, the preset recall is 0.9, meaning the first mutation detection module's recall in identifying gene mutation sites or in mutation detection is greater than or equal to 0.9, thereby reducing the probability of missing true mutation sites. In some embodiments, the first mutation detection module's recall in identifying gene mutation sites is greater than or equal to 0.95.

In some embodiments, the suspected mutation site acquisition module 310 is configured to align, by an alignment unit of the first mutation detection module, input sequencing data of the nucleic acid sample under test with sequencing data of a reference genome to obtain at least one gene variant site in the sequencing data of the nucleic acid sample different from the sequencing data of the reference genome; perform, by a feature extraction unit of the first mutation detection module, feature extraction on the at least one gene variant site to obtain at least one piece of first mutation feature data; and screen, by a mutation detection unit of the first mutation detection module, the suspected mutation site from the at least one gene variant site based on the at least one piece of first mutation feature data.

Due to the high recall of the first mutation detection module, this configuration ensures the completeness of the identified suspected mutation sites and makes certain false-positive mutation sites filtered out, thereby reducing the data amount of the suspected mutation sites and improving the gene mutation detection efficiency.

In this embodiment, the second mutation feature data is obtained from feature extraction through a second mutation detection module on each suspected mutation site. To distinguish the mutation feature data extracted by the second mutation detection module from the first mutation feature data extracted by the first mutation detection module, the mutation feature data extracted by the second mutation detection module is referred to as “second mutation feature data”.

For the sequencing data of the nucleic acid sample under test, variant sites represent genomic positions that differ from the reference genome and are present in the sequencing data. By way of example, the variant types of the suspected mutation sites include, but are not limited to, single nucleotide variation (SNV), insertion and deletion mutations, mismatch mutations, and tandem repeats. Single nucleotide polymorphism emphasizes the mutation frequency of suspected mutation sites in the population (for example, the mutation frequency >1%), where a single base at the suspected mutation site differs from the corresponding base in the reference genome. Single nucleotide variation does not emphasize the mutation frequency of suspected mutation sites in the population, where a single base at the suspected mutation site differs from the corresponding base in the reference genome. Insertion or deletion mutation represents the presence or absence of a new nucleic acid sequence fragment different from the reference genome and at the gene mutation site. Mismatch mutation represents the presence of mismatched base pairs at the gene mutation site. Tandem repeat represents the presence of one or more repeated nucleotides at the suspected mutation site.

Variant sites can be obtained by aligning sequencing data using a sequencing data alignment tool. By way of example, the alignment tool may be Bowtie2 or BLAST. Here the alignment tool is not limited and can be configured based on actual requirements.

Mutation sites refer to gene loci in a nucleic acid sample where base mutations cause sequence discrepancies with the reference genome, including single nucleotide variation and polynucleotide variant. It is to be understood that in embodiments of this application, mutation sites belong to the category of variant sites, with the ratio of mutation sites to variant sites ranging between 0 and 1.

To distinguish the mutation feature data extracted by the second mutation detection module from the first mutation feature data extracted by the first mutation detection module, the mutation feature data extracted by the second mutation detection module is referred to as “second mutation feature data”.

In embodiments of this application, both the first and second mutation detection modules are models used for detecting mutations in nucleic acid samples, but they operate on different mutation detection principles. In embodiments of this application, the second mutation detection module is introduced, and the mutation detection function of the second mutation detection module is used to extract features from the suspected mutation sites identified by the first mutation detection module. This is done to improve the precision of the target mutation detection model-trained using the extracted features as part of the features-in identifying the gene mutation sites, thereby ensuring that the proportion of true mutation sites among the suspected mutation sites identified by the first mutation detection module satisfies a predefined proportion.

In some embodiments, the precision of the second mutation detection module is greater than or equal to a preset precision, and the preset precision ensures that the precision of the target mutation detection model is greater than or equal to 0.8. By configuring the first mutation detection module to satisfy the preset recall, it is possible to improve the probability of identifying true mutation sites. Subsequently, the suspected mutation sites from the first mutation detection module are further analyzed by the second mutation detection module that satisfies the preset precision, and the extracted second mutation features are used as part of the input to the target mutation detection model, allowing the mutation detection results output by the target mutation detection model in the mutation detection result output module 340 to achieve both a high recall and a high precision.

In some embodiments, the preset precision is 0.9, meaning the second mutation detection module's precision in identifying gene mutation sites or in mutation detection is greater than or equal to 0.9. In this case, when identifying the gene mutation sites, the first mutation detection module has a recall greater than or equal to the preset recall, and the second mutation detection module has a precision greater than or equal to the preset precision. By configuring the first mutation detection module to satisfy the preset recall, it is possible to improve the probability of identifying true mutation sites. Subsequently, the suspected mutation sites from the first mutation detection module are further analyzed by the second mutation detection module that satisfies the preset precision, and the extracted second mutation features are used as part of the input to the target mutation detection model, allowing the mutation detection results output by the mutation detection result output module 340 to achieve both a high recall and a high precision.

In some embodiments, the recall of the first mutation detection module in identifying the gene mutation sites is greater than or equal to 0.9, and the precision of the second mutation detection module in identifying the gene mutation sites is also greater than or equal to 0.9. By using the first mutation detection module to reduce the probability of missing true mutation sites, it is possible to improve the likelihood of identifying the suspected mutation sites. Subsequently, the suspected mutation sites from the first mutation detection module are further analyzed by the second mutation detection module, thereby reducing the probability of missing false-positive mutation sites. Finally, the second mutation feature data extracted by the second mutation detection module are used as part of the input to the target mutation detection model, allowing the mutation detection results output by the target mutation detection model in the mutation detection result output module 340 to achieve both a high recall and a high precision.

In embodiments of this application, the recall and the precision may be obtained by testing the first mutation detection module and the second mutation detection module through a test set. The test set includes test sequencing data for testing nucleic acid samples.

In an embodiment, the first and second mutation detection modules may be screened from existing gene mutation detection model. By way of example, the first mutation detection module is implemented using the Varscan software, and the second mutation detection module is implemented using the Mutect2 software. In this case, when identifying gene mutation sites, the VarScan software exhibits a high recall, reaching up to 0.96, thereby increasing the probability of identifying true mutation sites. The suspected mutation sites obtained by VarScan are further analyzed using the Mutect2 software that has a precision greater than or equal to 0.9. The extracted second mutation features, together with the third mutation feature data extracted from the sequencing data based on the suspected mutation sites during sequencing data processing, are used as part of the input to the target mutation detection model. This enables the mutation detection results output by the target mutation detection model 340 to achieve both a high recall and a high precision. The results show that the target mutation detection model exhibits both a high recall and a high precision in mutation site detection.

In some embodiments, the third mutation feature data acquisition module 330 is configured to, for each suspected mutation site, acquire the processed sequencing data corresponding to the suspected mutation site from the total processed sequencing data and add the processed sequencing data to the third mutation feature data.

In some embodiments, the third mutation feature data acquisition module 330 is configured to, for each suspected mutation site, acquire the processed sequencing data corresponding to the suspected mutation site from the total processed sequencing data, perform feature screening on the processed sequencing data, and add the screened processed sequencing data to the third mutation feature data.

In embodiments of this application, the representation form of the mutation detection result may be, for example, a positive site or not, a grade, a score, or a probability. The grade may be a true mutation grade or a false-positive mutation grade. The score may be a true mutation score or a false-positive mutation score. The probability may be a true mutation rate or a false-positive mutation rate. Using the true mutation grade, true mutation score, or true mutation rate as an example, the higher the true mutation grade, true mutation score, or true mutation rate, the greater the likelihood that the suspected mutation site is a true mutation site. Conversely, the lower the true mutation grade, true mutation score, or true mutation rate, the lower the likelihood that the suspected mutation site is a true mutation site.

The representation form of the target mutation detection result is not limited and may be customized based on actual requirements.

In an optional embodiment, the mutation detection result indicates that the suspected mutation site is a positive site.

In an optional embodiment, the mutation detection result indicates that the suspected mutation site is a negative site.

The result of a positive site can be confirmed based on indicators such as the true mutation grade, true mutation score, and true mutation rate, but the indicators for confirming a suspected mutation site as a positive site are not limited thereto. Similarly, the result of a negative site can be confirmed based on indicators such as the false-positive mutation grade, false-positive mutation score, and false-positive mutation rate, but the indicators for confirming a suspected mutation site as a negative site are not limited thereto.

In some embodiments, inputting the second mutation feature data and the third mutation feature data into the pre-trained target mutation detection model and outputting the mutation detection result of each suspected mutation site includes inputting the second mutation feature data and the third mutation feature data into the pre-trained target mutation detection model to obtain a predicted value indicating that the suspected mutation site is positive; aligning the predicted value with a preset target value and outputting an alignment result; if the predicted value is greater than the preset target value, outputting the result that the suspected mutation site is positive; and if the predicted value is less than the preset target value, outputting the result that the suspected mutation site is negative.

Solutions of embodiments of this invention include acquiring a suspected mutation site of a nucleic acid sample under test by using the first mutation detection module with a recall greater than or equal to a preset recall, thereby ensuring the completeness of detecting the suspected mutation site; performing feature extraction on each suspected mutation site by using the second mutation detection module to obtain second mutation feature data; processing the sequencing data by using the sequencing data processing module to obtain overall processed sequencing data and screening processed sequencing data of each suspected mutation site from the overall processed sequencing data to obtain third mutation feature data; and inputting the second mutation feature data and the third mutation feature data into the pre-trained target mutation detection model and outputting a mutation detection result of each suspected mutation site. The target mutation detection model of this invention, based on the first mutation feature data with a high recall contribution, integrates the second mutation feature data extracted by the second mutation detection module different from the first mutation detection module and the third mutation feature data screened from sequencing data and related to suspected mutation features. The combination of the three types of mutation features addresses the problem of poor detection performance of traditional mutation detection methods or software systems and ensures both the high precision and the high recall of gene mutation detection.

In some embodiments, the first mutation detection module is implemented using the VarScan software, the second mutation detection module is implemented using the Mutect2 software or the Strelka2 software, and the sequencing data processing module is implemented using the bam-readcount software.

In this embodiment, the nucleic acid sample under test is from somatic cells. The suspected mutation site is determined based on first mutation feature data generated by the Varscan software upon mutation calling performed on sequencing data of the nucleic acid sample under test.

VarScan is a Java-based software used under Linux and used for detecting somatic mutations. With increasing sequencing depth (Read Depth (DP)), the recall of VarScan can reach approximately 0.96. However, after mutation detection using the VarScan software, it is still necessary to rely on methods such as genomic annotation, genetic annotation, and manual judgment to further validate the mutation detection results output by VarScan.

The second mutation feature data is obtained from feature extraction performed by Mutect2 or Strelka2 on the suspected mutation sites determined by VarScan. Mutect2 or Strelka2 is software for somatic mutation detection. Mutect2 employs a Bayesian algorithm based on a Hidden Markov Model and is primarily used to detect somatic single nucleotide variants and insertion and deletion mutations. Strelka2 performs rearrangement of gene variant sites of insertion and deletion mutations and conducts mutation detection based on a Bayesian probabilistic model and the mutation feature data of the rearranged gene variant sites. As the sequencing depth increases, the recall of Mutect2 or Strelka2 reaches a plateau value. This plateau value is influenced by the average error rate of the sequencing platform to which the sequencing technology belongs; therefore the plateau value is not very high. However, the precision of Mutect2 or Strelka2 can exceed 0.9.

In some embodiments, the second mutation feature data includes at least one of sequencing depth (Read Depth (DP)), number of mutation events (Number of Events (ECNT)), quality score of germline mutation (Germline Quality (GERMQ)), median quality score of reference bases (Median Base Quality (MBQ)), median quality score of mutant bases (Median Base Quality (MBQ)), median insert fragment length of reference bases (Median Fragment Length by Allele (MFRL)), median insert fragment length of mutant bases (Median Fragment Length by Allele (MFRL)), median mapping quality score of reference bases (Median Mapping Quality (MMQ)), median mapping quality score of mutant bases (Median Mapping Quality (MMQ)), Median Position (MPOS), Negative log 10 odds of artifact in normal with same allele fraction as tumor (Negative Allele Fraction in Normal (NALOD)), Normal log 10 likelihood ratio of diploid het or hom alt genotypes (Normal Log Odds (NLOD)), or Log 10 likelihood ratio score of variant existing versus not existing (Tumor Log Odds (TLOD)).

In some embodiments, the second mutation feature data includes at least one of sequencing depth (Read Depth (DP)), number of mutation events (Number of Events (ECNT)), median quality score of reference bases (Median Base Quality (MBQ)), median quality score of mutant bases (Median Base Quality (MBQ)), median insert fragment length of reference bases (Median Fragment Length by Allele (MFRL)), median insert fragment length of mutant bases (Median Fragment Length by Allele (MFRL)), median mapping quality score of reference bases (Median Mapping Quality (MMQ)), median mapping quality score of mutant bases (Median Mapping Quality (MMQ)), Median Position (MPOS), Negative log 10 odds of artifact in normal with same allele fraction as tumor (Negative Allele Fraction in Normal (NALOD)), Normal log 10 likelihood ratio of diploid het or hom alt genotypes (Normal Log Odds (NLOD)), or Log 10 likelihood ratio score of variant existing versus not existing (Tumor Log Odds (TLOD)).

In some embodiments, the second mutation feature data includes sequencing depth (Read Depth (DP)), number of mutation events (Number of Events (ECNT)), median quality score of reference bases (Median Base Quality (MBQ)), median quality score of mutant bases (Median Base Quality (MBQ)), median insert fragment length of reference bases (Median Fragment Length by Allele (MFRL)), median insert fragment length of mutant bases (Median Fragment Length by Allele (MFRL)), median mapping quality score of reference bases (Median Mapping Quality (MMQ)), median mapping quality score of mutant bases (Median Mapping Quality (MMQ)), Median Position (MPOS), Negative log 10 odds of artifact in normal with same allele fraction as tumor (Negative Allele Fraction in Normal (NALOD)), Normal log 10 likelihood ratio of diploid het or hom alt genotypes (Normal Log Odds (NLOD)), and Log 10 likelihood ratio score of variant existing versus not existing (Tumor Log Odds (TLOD)).

The definition of the second mutation feature data is as previously described. To save space, the details are not repeated here.

The bam-readcount software processes genomic sequencing data to obtain a readcount file. The third mutation feature data in the readcount file provides comprehensive sequencing and alignment information for suspected mutation sites, serving as critical feature evidence for gene mutation detection of suspected mutation sites.

In some optional embodiments, the third mutation feature data includes at least one of average mapping quality value (avg_mapping_quality (amq)), average base quality value (avg_basequality (ab)), average position as fraction (avg_pos_as_fraction (apf)), average number of mismatch bases as fraction (avg_num_mismatches_as_fraction (anmf)), or average sum of quality score of mismatch bases (avg_sum_mismatch_qualitie (asmq)).

In some embodiments, the third mutation feature data includes at least one of average mapping quality value (avg_mapping_quality (amq)), average base quality value (avg_basequality (ab)), or average position as fraction (avg_pos_as_fraction (apf)).

In some embodiments, the third mutation feature data includes average mapping quality value (avg_mapping_quality (amq)), average base quality value (avg_basequality (ab)), and average position as fraction (avg_pos_as_fraction (apf)).

The definition of the third mutation feature data is as previously described. To save space, the details are not repeated here.

In the technical solution of this embodiment, the first mutation detection module is implemented using the Varscan software, and the second mutation detection module is implemented using the Mutect2 or Strelka2 software, where Varscan satisfies the constraint of a recall ≥0.9, and Mutect2 satisfies the constraint of a precision ≥0.9. This improves both the recall and precision of the target mutation detection model.

Based on the preceding embodiments, the target mutation detection model in the embodiments of this application is pre-trained. In some embodiments, the apparatus further includes a target mutation detection model training module, and the target mutation detection model training module includes a training mutation site acquisition unit, a predicted mutation detection result output unit, and a target mutation detection model determination unit.

The training mutation site acquisition unit is configured to acquire a training mutation site of a training nucleic acid sample from the first mutation detection module.

The predicted mutation detection result output unit is configured to input second training feature data and third training feature data of the training mutation site into an initial mutation detection model to be trained, to obtain output of a predicted mutation detection result of the training mutation site, where the second training feature data is feature data obtained from feature extraction of the second mutation detection module on the training mutation site, and the third training feature data is feature data obtained from processing of the sequencing data processing module on training sequencing data.

The target mutation detection model determination unit is configured to train the initial mutation detection model based on the predicted mutation detection result and a standard mutation detection result corresponding to the training mutation site to obtain the target mutation detection model trained.

In the training mutation site acquisition unit, the training nucleic acid sample is a nucleic acid sample containing a known gene mutation site, such as a standard nucleic acid sample. That the gene mutation site is known includes that nucleic acid sequences of the training nucleic acid sample are known, and the gene mutation site can be determined according to the known sequences or includes that for a gene mutation site in the training nucleic acid sample, a position of the site in a nucleic acid sequence and a mutation type of the site can be determined. The training nucleic acid sample may contain one known gene mutation site or contain two or more known gene mutation sites.

Generally, an increase in the number of training nucleic acid samples facilitates an improvement in the accuracy of the model during training of the initial mutation detection model. Nucleic acid samples with known gene mutation sites are difficult to collect in practice. Therefore, in some implementations, the nucleic acid sample with the gene mutation site, that is, the training nucleic acid sample, may be simulated based on reference standards.

As an embodiment, the training nucleic acid sample in the embodiments of this application is a simulated tumor reference standard. Correspondingly, the training sequencing data is sequencing data of the simulated tumor reference standard, that is, simulated tumor reference standard sequencing data. The simulated tumor reference standard sequencing data in the embodiments of this application is a set of data artificially constructed by computational methods. This set of data stimulates a sequencing result of an actual tumor sample. The simulated tumor reference standard sequencing data is based on modifications or processing of existing sequencing data from cells or tissues to introduce specific variant or mutation features, thereby simulating the genomic features of tumor cells. It is to be noted that the simulated tumor reference standard sequencing data do not directly originate from real tumor sample sequencing, but are synthesized by researchers through algorithm design according to known tumor genomic features and the characteristics of sequencing technology. These data include simulated somatic mutations and other tumor-associated genomic features. Such simulated data can be used for evaluating and optimizing the sequencing technology and bioinformatics analysis algorithms and workflows and training and testing machine learning models.

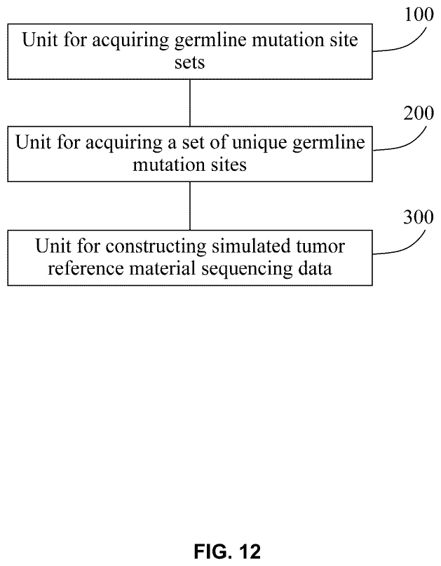

In another aspect of this application, this application provides an apparatus for constructing the simulated tumor reference standard sequencing data. Referring to FIG. 12, the apparatus includes a unit 100 for acquiring germline mutation site sets, a unit 200 for acquiring a set of unique germline mutation sites, and a unit 300 for constructing the simulated tumor reference standard sequencing data.

The unit 100 is configured to, for a targeted region, acquire a first germline mutation site set from a first human genome reference standard and a second germline mutation site set from a second human genome reference standard.

In some examples of this application, the targeted region is determined based on specific high incidence regions of tumor somatic mutations. Germline mutation sites obtained based on the targeted region constitute a set of targeted germline mutation sites.

In some examples of this application, the first human genome reference standard and the second human genome reference standard are each independently selected from at least one of HG001, HG002, HG003, HG004, or HG005, and the first human genome reference standard and the second human genome reference standard are different.

In some examples of this application, germline mutations, such as SNVs or indels, are selected from high confidence germline mutations.

The unit 200 is configured to select, from the first germline mutation site set, the set of unique germline mutation sites relative to the second germline mutation site set.

In some examples of this application, after the unit 200 and before the unit 300, the apparatus further includes an optimization unit configured to preprocess the set of unique germline mutation sites.

In some examples of this application, the optimization unit includes a first optimization subunit configured to remove, from the set of unique germline mutation sites, the same low confidence germline mutation site in the set of unique germline mutation sites and the second germline mutation site set.

In some other examples of this application, the optimization unit further includes a second optimization subunit configured to remove at least one unique germline mutation site from the set of unique germline mutation sites based on position relationships between the unique germline mutation sites in the set of unique germline mutation sites. Optionally, the at least one unique germline mutation site is removed from the set of unique germline mutation sites based on the distances between the unique germline mutation sites in the set of unique germline mutation sites. Specifically, the distances between the unique germline mutation sites in the set of unique germline mutation sites are determined; and when a distance of the distances between the unique germline mutation sites is less than a predetermined distance threshold, the at least one unique germline mutation site is removed from the set of unique germline mutation sites so that the distances between the remaining unique germline mutation sites are greater than or equal to the predetermined distance threshold.

In some examples of this application, the predetermined distance threshold is related to a sequencing read length of a sequencing platform for acquiring the sequencing data. If unique germline mutation sites for simulation are too close to each other, one sequencing read generated through sequencing may contain more than one unique germline mutation site for simulation, resulting in mutual interference during simulation. Therefore, the distance threshold for the unique germline mutation sites for simulation is not less than 250 bp. Optionally, the distance threshold is 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, or 400 bp.

The unit 300 is configured to acquire sequencing data of the second human genome reference standard and sequencing data of the first human genome reference standard, and for at least one preset simulated somatic mutation site, replace sequencing data corresponding to the at least one preset simulated somatic mutation site in the sequencing data of the second human genome reference standard with sequencing data corresponding to the at least one preset simulated somatic mutation site in the sequencing data of the first human genome reference standard according to a predetermined replacement ratio to obtain the simulated tumor reference standard sequencing data containing simulated somatic mutations, where the at least one preset simulated somatic mutation site is selected from the set of unique germline mutation sites.

In some examples of this application, the predetermined replacement ratio is related to a mutation frequency of the preset simulated somatic mutation site.

In some examples of this application, the units are connected, where “connected” refers to “physically connected” or “connected via a network”.

The apparatus can generate the simulated tumor reference standard sequencing data efficiently and accurately.

It is to be understood that the apparatus for constructing the simulated tumor reference standard sequencing data may serve as a function module in the gene mutation detection apparatus, such as a simulated tumor reference standard sequencing data construction module.

The training sequencing data of the training nucleic acid sample refers to nucleic acid sequence data of the training nucleic acid sample. The nucleic acid sequence data of the training nucleic acid sample may be all sequence data of the training nucleic acid sample, partial sequence data of the training nucleic acid sample including gene variant sites, or even types of bases at a gene mutation site. The types of bases at the gene mutation site include a type of base before the mutation and a type of base after the mutation.

In an embodiment of this application, the first mutation detection module performs feature extraction on the training sequencing data of the training nucleic acid sample and acquires the training mutation site based on extracted features. For each training nucleic acid sample, the software may acquire one training mutation site or two or more training mutation sites. The first mutation detection module is as described above, and for the acquisition of the training mutation site of the training nucleic acid sample by the first mutation detection module, reference is made to the content of “acquiring a suspected mutation site of a nucleic acid sample under test”. To save space, the details are not repeated here. Further, secondary confirmation may be performed on the “suspected mutation site” obtained by the preceding method to confirm whether the suspected mutation site is a true or false positive.

In the predicted mutation detection result output unit, the second training feature data is the feature data obtained from the feature extraction of the second mutation detection module on each training mutation site, and the third training feature data is acquired as follows: the sequencing data processing module processes the training sequencing data to obtain total processed training sequencing data and screens processed sequencing data of each training mutation site from the total processed training sequencing data to obtain the third training feature data. The second mutation detection module and the sequencing data processing module are not described in detail here.

In some embodiments, the second training feature data includes the preceding second mutation feature data, and the third training feature data includes the preceding third mutation feature data, which are not described in detail here.

For each training mutation site, the second training feature data acquired by the second mutation detection module and the third training feature data acquired by the sequencing data processing module are acquired and inputted into the initial mutation detection model to be trained, and training is performed so that the output of the predicted mutation detection result of the training mutation site is obtained. In an embodiment of this application, the initial mutation detection model is an untrained initial model of the target mutation detection model.

In the target mutation detection model determination unit, the initial mutation detection model is trained based on the predicted mutation detection result obtained by the predicted mutation detection result output unit and the standard mutation detection result corresponding to the training mutation site acquired by the training mutation site acquisition unit, to obtain the target mutation detection model trained. It is to be understood that for the same training mutation site, a one-to-one correspondence relationship exists between the predicted mutation detection result obtained by the predicted mutation detection result output unit and the standard mutation detection result corresponding to the training mutation site from the training mutation site acquisition unit.

In an embodiment of this application, a model algorithm of the target mutation detection model is not strictly limited. In some embodiments, the model algorithm of the target mutation detection model is a gradient boosting model. For example, the model algorithm of the target mutation detection model includes, but is not limited to, a logical classification model, a decision tree, a support vector machine, random forests, an AdaBoost model, an XGBoost model, or a deep confidence network, which may be customized according to actual requirements.

Specifically, the target mutation detection model determination unit is configured to determine a loss function according to the predicted mutation detection result and the standard mutation detection result corresponding to the training mutation site, adjust a model parameter of the initial mutation detection model according to the loss function until a preset ending condition is satisfied, and use the initial mutation detection model in a current iteration process as the target mutation detection model trained.

For example, a type of the loss function includes, but is not limited to, a square loss function, a log loss function, an exponential loss function, a mean squared error loss function, a logistic regression loss function, a Huber loss function, a cross-entropy loss function, or a Kullback-Leibler divergence loss function. The type of the loss function is not limited here and may be customized according to actual requirements.

Based on the preceding embodiments, optionally, the apparatus further includes a target mutation detection model verification module, which is configured to, before the initial mutation detection model in the current iteration process is used as the target mutation detection model verified, acquire a verification mutation site in verification sequencing data of a verification nucleic acid sample; acquire second verification feature data and third verification feature data of the verification mutation site extracted by the second mutation detection module and the sequencing data processing module, respectively, and input the second verification feature data and the third verification feature data into the initial mutation detection model in the current iteration process to obtain the output of a verification mutation detection result of the verification mutation site; and determine model performance of the initial mutation detection model according to the verification mutation detection result, and in the case where the model performance satisfies a preset performance condition, use the initial mutation detection model in the current iteration process as the target mutation detection model verified.

For example, the model performance includes, but is not limited to, at least one of the recall, the precision, an F1 score, an accuracy, or the area under an ROC curve. The model performance is not limited here and may be customized according to actual requirements.

Based on the preceding embodiments, the apparatus further includes a hyperparameter adjustment module, which is configured to, in the case the model performance does not satisfy the preset performance condition, adjust a hyperparameter of the initial mutation detection model, and return to the step of acquiring the training mutation site in the training sequencing data of the training nucleic acid sample.

In some embodiments, an architecture of the target mutation detection model is a classification model; and the apparatus further includes a second mutation feature data screening module and a third mutation feature data screening module.

Before the second mutation feature data and the third mutation feature data are input into the pre-trained target mutation detection model and the mutation detection result of each suspected mutation site is output, the second mutation feature data screening module is configured to screen the second mutation feature data according to feature weights corresponding to at least two second mutation features in the second mutation feature data.

The third mutation feature data screening module is configured to screen the third mutation feature data according to feature weights corresponding to at least two third mutation features in the third mutation feature data. Each feature weight is determined by the target mutation detection model during the last iterative training.

In some embodiments, the second mutation feature data screening module is configured to screen second mutation features whose feature weights are greater than a first weight threshold as the screened second mutation features. For example, the first weight threshold may be 0.001, and the first weight threshold is not limited here and may be customized according to actual situations.

In some embodiments, the second mutation feature data screening module is configured to sort the at least two second mutation features according to the feature weights corresponding to the at least two second mutation features, and determine the screened second mutation features according to a sorting result and a first selection number or a first selection proportion. For example, the first selection number may be 5, and the first selection proportion may be 50%. The first selection number or the first selection proportion is not limited here and may be customized according to actual requirements.

In some embodiments, the third mutation feature data screening module is configured to screen third mutation features whose feature weights are greater than a second weight threshold as the screened third mutation features. For example, the second weight threshold may be 0.0001, and the second weight threshold is not limited here and may be customized according to actual situations.

In some embodiments, the third mutation feature data screening module is configured to sort the at least two third mutation features according to the feature weights corresponding to the at least two third mutation features, and determine the screened third mutation features according to a sorting result and a second selection number or a second selection proportion. For example, the second selection number may be 10, and the second selection proportion may be 60%. The second selection number or the second selection proportion is not limited here and may be customized according to actual requirements.

Such an arrangement has the following advantages: a data volume of mutation features input into the target mutation detection model can be reduced, and a computational amount of the target mutation detection model can be increased, thereby improving the efficiency of gene mutation detection.

According to the technical solutions of this embodiment, at least two pre-trained reference mutation detection models are acquired, and the first mutation detection module and the second mutation detection module are determined from the at least two reference mutation detection models according to a recall and a precision corresponding to each reference mutation detection model, thereby solving the problem of manual screening of the first mutation detection module and the second mutation detection module and improving the screening efficiency of the first mutation detection module and the second mutation detection module while ensuring that the first mutation detection module and the second mutation detection module satisfy performance constraint conditions.

The gene mutation detection apparatus according to the embodiments of this invention may perform the gene mutation detection method according to any embodiment of this invention and has function modules and beneficial effects corresponding to the performed method.

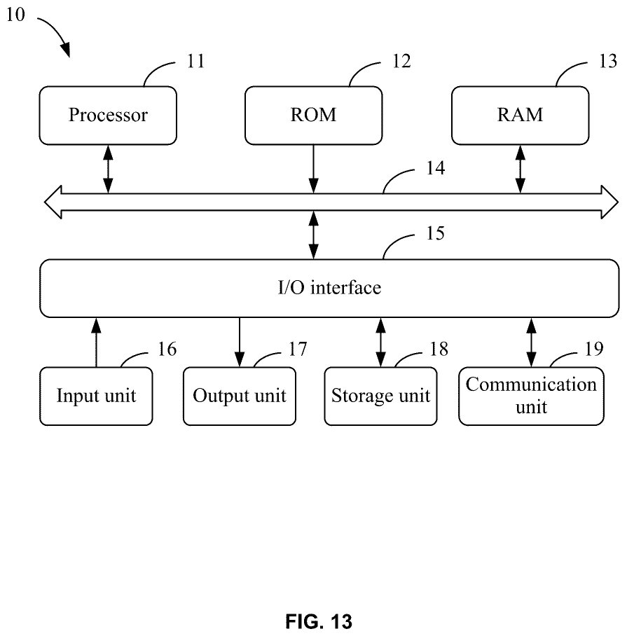

FIG. 13 is a diagram illustrating the structure of an electronic device according to an embodiment of this invention. The electronic device 10 is intended to represent various forms of digital computers, for example, a laptop computer, a desktop computer, a worktable, a server, a blade server, a mainframe computer, and another applicable computer. The electronic device may also represent various forms of mobile apparatuses, for example, a personal digital assistant, a cellphone, a smartphone, a wearable device (such as a helmet, glasses, or a watch), and another similar computing apparatus. Herein the shown components, the connections and relationships between these components, and the functions of these components are illustrative only and are not intended to limit the implementation of this invention as described and/or claimed herein.

As shown in FIG. 13, the electronic device 10 includes at least one processor 11 and a memory communicatively connected to the at least one processor 11, such as a read-only memory (ROM) 12 and a random-access memory (RAM) 13. The memory stores a computer program executable by the at least one processor. The at least one processor 11 can perform various appropriate actions and processing according to a computer program stored in the read-only memory (ROM) 12 or a computer program loaded into the random-access memory (RAM) 13 from a storage unit 18. Various programs and data required for the operation of the electronic device 10 may also be stored in the RAM 13. The at least one processor 11, the ROM 12, and the RAM 13 are connected to each other through a bus 14. An input/output (I/O) interface 15 is also connected to the bus 14.

Multiple components in the electronic device 10 are connected to the I/O interface 15. The multiple components include an input unit 16 such as a keyboard or a mouse, an output unit 17 such as various types of display or speaker, the storage unit 18 such as a magnetic disk or an optical disk, and a communication unit 19 such as a network card, a modem, or a wireless communication transceiver. The communication unit 19 allows the electronic device 10 to exchange information/data with other devices over a computer network such as the Internet and/or various telecommunications networks.

The at least one processor 11 may be various general-purpose and/or special-purpose processing components having processing and computing capabilities. Examples of a processor 11 include, but are not limited to, a central processing unit (CPU), a graphics processing unit (GPU), a special-purpose artificial intelligence (AI) computing chip, a processor executing machine learning models and algorithms, a digital signal processor (DSP), and any appropriate processor, controller, or microcontroller. The processor 11 performs various preceding methods and processing, such as the gene mutation detection method.

In some embodiments, the gene mutation detection method in the preceding embodiments may be implemented as a computer program tangibly included in a computer-readable storage medium such as the storage unit 18. In some embodiments, part or all of the computer programs may be loaded and/or installed onto the electronic device 10 via the ROM 12 and/or the communication unit 19. When the computer programs are loaded to the RAM 13 and executed by the processor 11, one or more steps of the gene mutation detection method for a light curtain may be performed. Alternatively, in other embodiments, the processor 11 may be configured, in any other suitable manner (for example, by means of firmware), to perform the gene mutation detection method.

The various embodiments of the systems and techniques described herein may be implemented in one of or a combination of at least two of the following systems: digital electronic circuitry, integrated circuitry, a field-programmable gate array (FPGA), an application-specific integrated circuit (ASIC), application specific standard parts (ASSP), a system on a chip (SoC), a complex programmable logic device (CPLD), computer hardware, firmware, software, and/or a combination thereof. The various embodiments may include implementations in one or more computer programs. The one or more computer programs are executable and/or interpretable on a programmable system including at least one programmable processor. A programmable processor may be a special-purpose or general-purpose programmable processor for receiving data and instructions from a memory system, at least one input apparatus, and at least one output apparatus and transmitting data and instructions to the memory system, the at least one input apparatus, and the at least one output apparatus.

Computer programs for implementation of the gene mutation detection method of this invention may be written in one programming language or any combination of multiple programming languages. The computer programs may be provided for a processor of a general-purpose computer, a special-purpose computer, or another programmable data processing apparatus to enable functions/operations specified in a flowchart and/or a block diagram to be implemented when the computer programs are executed by a processor. The computer programs may be executed entirely on a machine, partly on a machine, as a stand-alone software package, partly on a machine and partly on a remote machine, or entirely on a remote machine or a server.

In the context of this application, the computer-readable storage medium may be a tangible medium that may include or store a computer program used by or in conjunction with an instruction execution system, apparatus, or device. The computer-readable storage medium may include, but is not limited to, an electronic, magnetic, optical, electromagnetic, infrared, or semiconductor system, apparatus, or device, or any appropriate combination thereof. Alternatively, the computer-readable storage medium may be a machine-readable storage medium. Examples of the machine-readable storage medium may include an electrical connection based on one or more wires, a portable computer disk, a hard disk, a random-access memory (RAM), a read-only memory (ROM), an erasable programmable read-only memory (EPROM), a flash memory, an optical fiber, a portable compact disk read-only memory (CD-ROM), an optical memory device, a magnetic memory device or any suitable combination thereof.

In order that interaction with a user is provided, the systems and techniques described herein may be implemented in the terminal device. The terminal device has a display apparatus (for example, a cathode-ray tube (CRT) or a liquid-crystal display (LCD) monitor) for displaying information to the user; and a keyboard and a pointing apparatus (for example, a mouse or a trackball) through which the user can provide input for the terminal device. Other types of apparatuses may also be used for providing interaction with the user. For example, feedback provided for the user may be sensory feedback in any form (for example, visual feedback, auditory feedback, or tactile feedback); and input from the user may be received in any form (including acoustic input, voice input, or tactile input).

The systems and techniques described herein may be implemented in a computing system including a back-end component (for example, a data server), a computing system including a middleware component (for example, an application server), a computing system including a front-end component (for example, a client computer having a graphical user interface or a web browser through which the user can interact with embodiments of the systems and techniques described herein), or a computing system including any combination of such back-end, middleware, or front-end components. Components of a system may be interconnected by any form or medium of digital data communication (for example, a communication network). Examples of the communication network include a local area network (LAN), a wide area network (WAN), a blockchain network, and the Internet.

The computing system may include clients and servers. A client and a server are generally remote from each other and typically interact through a communication network. The relationship between the client and the server arises by virtue of computer programs running on respective computers and having a client-server relationship to each other. The server may be a cloud server, also referred to as a cloud computing server or a cloud host. As a host product in a cloud computing service system, the server solves the defects of difficult management and weak service scalability in conventional physical host and virtual private server (VPS) services.

It is to be understood that various forms of the preceding flows may be used with steps reordered, added, or deleted. For example, the steps described in this invention may be performed in parallel, in sequence, or in a different order as long as the desired result of the technical solutions provided in this invention can be achieved. The execution sequence of these steps is not limited herein.

The scope of this invention is not limited to the preceding embodiments. It is to be understood by those skilled in the art that various modifications, combinations, subcombinations, and substitutions may be made according to design requirements and other factors. Any modification, equivalent substitution, or improvement made within the spirit and principle of this invention fall within the scope of this invention.

What is claimed is:

1. A gene mutation detection method, comprising:
acquiring a suspected mutation site of a nucleic acid sample under test, wherein the suspected mutation site is determined based on first mutation feature data generated by a first mutation detection module upon mutation calling performed on sequencing data of the nucleic acid sample under test, and a recall at which the first mutation detection module identifies gene mutation sites is greater than or equal to a preset recall;
performing, by a second mutation detection module, feature extraction on each suspected mutation site to obtain second mutation feature data;
processing, by a sequencing data processing module, the sequencing data to obtain overall processed sequencing data and screening processed sequencing data of each suspected mutation site from the overall processed sequencing data to obtain third mutation feature data; and
inputting the second mutation feature data and the third mutation feature data into a pre-trained target mutation detection model and outputting a mutation detection result of each suspected mutation site.

2. The method of claim 1, wherein
the second mutation feature data comprises at least one of sequencing depth, number of mutation events, quality score of germline mutation, median quality score of reference bases, median quality score of mutant bases, median insert fragment length of reference bases, median insert fragment length of mutant bases, median mapping quality score of reference bases, median mapping quality score of mutant bases, median position, Negative log 10 odds of artifact in normal with same allele fraction as tumor (NALOD), Normal log 10 likelihood ratio of diploid het or hom alt genotypes (NLOD), or Log 10 likelihood ratio score of variant existing versus not existing (TLOD), wherein
the sequencing depth represents a number of times a site is covered by reads;
the number of mutation events represents a number of observed variant events at an identified suspected mutation site;
the quality score of germline mutation represents a quality score at which an identified suspected mutation site is not a germline variant and indicates a probability that the suspected mutation site is not a germline variant;
the median quality score of reference bases represents a median quality score of bases that match a reference genome base at an identified suspected mutation site;
the median quality score of mutant bases represents a median quality value of mutant bases corresponding to an identified suspected mutation;
the median insert fragment length of reference bases represents a median insert fragment length of paired-end reads whose bases, at a genomic position corresponding to an identified suspected mutation site, match a reference genome base and thus represent an unmutated allele;
the median insert fragment length of mutant bases represents a median insert fragment length of paired-end reads whose bases, at a site corresponding to an identified suspected mutation, exhibit a same mutation type and thus constitute a mutant allele;
the median mapping quality score of reference bases represents a median mapping quality value of bases that match a reference genome base and correspond to an alternate allele of an identified suspected mutation site;
the median mapping quality score of mutant bases represents a median mapping quality value of mutant bases corresponding to an identified suspected mutation;
the median position represents a median position from an identified suspected mutation site to ends of reads containing the identified suspected mutation site;
the Negative log 10 odds of artifact in normal with same allele fraction as tumor (NALOD) represents a negative logarithm of a probability that a mutation identical to an identified suspected mutation, with a same frequency, and in sequencing data of a non-mutant sample is a false positive;
the Normal log 10 likelihood ratio of diploid het or hom alt genotypes (NLOD) represents a logarithm of a likelihood ratio that an identified suspected mutation site in sequencing data of a non-mutant sample is a true germline mutation (heterozygous or homozygous); and
the Log 10 likelihood ratio score of variant existing versus not existing (TLOD) represents a logarithm of a likelihood ratio that a suspected mutation site is a true somatic mutation; and
the third mutation feature data comprises at least one of average mapping quality value, average base quality value, average position as fraction, average number of mismatch bases as fraction, or average sum of quality score of mismatch bases, wherein
the average mapping quality value is an average of mapping quality values of all detected mutant bases corresponding to a determined gene locus in a reference genome;
the average base quality value is an average of base quality values of bases corresponding to an identified suspected mutation site across reads;
the average position as fraction is an average position as fraction of base positions at a suspected mutation site relative to nucleic acid fragment reference base positions across reads containing a same suspected mutation site;
the average number of mismatch bases as fraction is an average number fraction of bases different from a reference genome across reads;
the average sum of quality score of mismatch bases is an average quality value of bases different from a human reference genome across reads corresponding to an identified suspected mutation site.

3. The method of claim 2, wherein the second mutation feature data comprises at least one of sequencing depth, number of mutation events, median quality score of reference bases, median quality score of mutant bases, median insert fragment length of reference bases, median insert fragment length of mutant bases, median mapping quality score of reference bases, median mapping quality score of mutant bases, median position, Negative log 10 odds of artifact in normal with same allele fraction as tumor (NALOD), Normal log 10 likelihood ratio of diploid het or hom alt genotypes (NLOD), or Log 10 likelihood ratio score of variant existing versus not existing (TLOD),
the third mutation feature data comprises at least one of average mapping quality value, average base quality value, or average position as fraction.

4. The method of claim 2, wherein the preset recall is 0.9, and the recall at which the first mutation detection module identifies the gene mutation sites is greater than or equal to 0.95.

5. The method of claim 1, wherein
screening the processed sequencing data of each suspected mutation site from the overall processed sequencing data to obtain the third mutation feature data comprises:
for each suspected mutation site, acquiring the processed sequencing data corresponding to the suspected mutation site from the total processed sequencing data and adding the processed sequencing data to the third mutation feature data; or
screening the processed sequencing data of each suspected mutation site from the overall processed sequencing data to obtain the third mutation feature data comprises:
for each suspected mutation site, acquiring the processed sequencing data corresponding to the suspected mutation site from the total processed sequencing data, performing feature screening on the processed sequencing data, and adding the screened processed sequencing data to the third mutation feature data.

6. The method of claim 1, wherein
the mutation detection result is a result that the suspected mutation site is positive or a result that the suspected mutation site is negative; and inputting the second mutation feature data and the third mutation feature data into the pre-trained target mutation detection model and outputting the mutation detection result of each suspected mutation site comprises:
inputting the second mutation feature data and the third mutation feature data into the pre-trained target mutation detection model to obtain a predicted value indicating that the suspected mutation site is positive;
aligning the predicted value with a preset target value and outputting an alignment result;
if the predicted value is greater than the preset target value, outputting the result that the suspected mutation site is positive; and
if the predicted value is less than the preset target value, outputting the result that the suspected mutation site is negative.

7. The method of claim 1, wherein acquiring the suspected mutation site of the nucleic acid sample under test comprises:
aligning, by an alignment unit of the first mutation detection module, input sequencing data of the nucleic acid sample under test with sequencing data of a reference genome to obtain at least one gene variant site in the sequencing data of the nucleic acid sample different from the sequencing data of the reference genome;
performing, by a feature extraction unit of the first mutation detection module, feature extraction on the at least one gene variant site to obtain at least one piece of first mutation feature data; and
screening, by a mutation detection unit of the first mutation detection module, the suspected mutation site from the at least one gene variant site based on the at least one piece of first mutation feature data.

8. The method of claim 7, wherein a precision of the second mutation detection module is greater than or equal to a preset precision, and the preset precision ensures that a precision of the target mutation detection model is greater than or equal to 0.8.

9. The method of claim 1, wherein an architecture of the first mutation detection module is Varscan software, and an architecture of the second mutation detection module is Mutect2 software.

10. The method of claim 1, further comprising:
acquiring, by the first mutation detection module, a training mutation site of a training nucleic acid sample, wherein the training nucleic acid sample is a nucleic acid sample containing a known mutation site, and the training mutation site is determined by first training feature data obtained from mutation detection of the first mutation detection module on training sequencing data of the training nucleic acid sample;
inputting second training feature data and third training feature data of the training mutation site into an initial mutation detection model to be trained, to obtain output of a predicted mutation detection result of the training mutation site, wherein the second training feature data is feature data obtained from feature extraction of the second mutation detection module on each training mutation site, and the third training feature data is acquired as follows: the sequencing data processing module processes the training sequencing data to obtain total processed training sequencing data and screens processed sequencing data of each training mutation site from the total processed training sequencing data to obtain the third training feature data; and
training the initial mutation detection model based on the predicted mutation detection result and a standard mutation detection result corresponding to the training mutation site to obtain the target mutation detection model trained.

11. The method of claim 1, wherein the training nucleic acid sample is a simulated tumor reference standard, and the training sequencing data is simulated tumor reference standard sequencing data.

12. The method of claim 11, wherein the simulated tumor reference standard sequencing data is obtained as follows:
(a) for a targeted region, acquiring a first germline mutation site set from a first human genome reference standard and a second germline mutation site set from a second human genome reference standard;
(b) selecting, from the first germline mutation site set, a set of unique germline mutation sites relative to the second germline mutation site set; and
(c) acquiring sequencing data of the second human genome reference standard and sequencing data of the first human genome reference standard, and for at least one preset simulated somatic mutation site, in accordance with a predetermined replacement ratio, replacing sequencing data originating from the at least one preset simulated somatic mutation site in the sequencing data of the second human genome reference standard with sequencing data originating from the at least one preset simulated somatic mutation site in the sequencing data of the first human genome reference standard, thereby obtaining the simulated tumor reference standard sequencing data containing simulated somatic mutations, wherein the at least one preset simulated somatic mutation site is selected from the set of unique germline mutation sites.

13. The method of claim 12, before performing step (c), further comprising preprocessing the set of unique germline mutation sites.

14. The method of claim 13, wherein the preprocessing comprises removing at least one unique germline mutation site from the set of unique germline mutation sites based on position relationships between the unique germline mutation sites in the set of unique germline mutation sites.

15. The method of claim 14, wherein the at least one unique germline mutation site is removed from the set of unique germline mutation sites based on distances between the unique germline mutation sites in the set of unique germline mutation sites.

16. The method of claim 13, wherein the preprocessing comprises:
determining distances between the unique germline mutation sites in the set of unique germline mutation sites; and
when a distance of the distances between the unique germline mutation sites is less than a predetermined distance threshold, removing at least one unique germline mutation site from the set of unique germline mutation sites so that distances between remaining unique germline mutation sites in the set of unique germline mutation sites are greater than or equal to the predetermined distance threshold.

17. The method of claim 13, wherein
an architecture of the target mutation detection model is a classification model; and
before inputting the second mutation feature data and the third mutation feature data into the pre-trained target mutation detection model and outputting the mutation detection result of each suspected mutation site, the method further comprises:
screening the second mutation feature data according to feature weights corresponding to at least two second mutation features in the second mutation feature data; and
screening the third mutation feature data according to feature weights corresponding to at least two third mutation features in the third mutation feature data;
wherein each feature weight is determined by the target mutation detection model during last iterative training.

18. The method of claim 1, wherein the nucleic acid sample under test has a mutation frequency less than or equal to 1.5%.

19. A gene mutation detection apparatus, comprising:
a suspected mutation site acquisition module configured to acquire a suspected mutation site of a nucleic acid sample under test, wherein the suspected mutation site is determined based on first mutation feature data generated by a first mutation detection module upon mutation calling performed on sequencing data of the nucleic acid sample under test, and a recall at which the first mutation detection module identifies gene mutation sites is greater than or equal to a preset recall;
a second mutation feature data acquisition module configured to perform feature extraction through a second mutation detection module on each suspected mutation site to obtain second mutation feature data;
a third mutation feature data acquisition module configured to process the sequencing data through a sequencing data processing module to obtain overall processed sequencing data and screen processed sequencing data of each suspected mutation site from the overall processed sequencing data to obtain third mutation feature data; and
a mutation detection result output module configured to input the second mutation feature data and the third mutation feature data into a pre-trained target mutation detection model and output a mutation detection result of each suspected mutation site.

20. An electronic device, comprising:
at least one processor; and
a memory communicatively connected to the at least one processor;
wherein the memory stores a computer program executable by the at least one processor to enable the at least one processor to perform a gene mutation detection method, wherein the gene mutation detection method comprises:
acquiring a suspected mutation site of a nucleic acid sample under test, wherein the suspected mutation site is determined based on first mutation-feature data generated by a first mutation-detection module upon mutation calling performed on sequencing data of the nucleic-acid sample under test, and a recall at which the first mutation detection module identifies gene mutation sites is greater than or equal to a preset recall;
performing, by a second mutation detection module, feature extraction on each suspected mutation site to obtain second mutation feature data;
processing, by a sequencing data processing module, the sequencing data to obtain overall processed sequencing data and screening processed sequencing data of each suspected mutation site from the overall processed sequencing data to obtain third mutation feature data; and
inputting the second mutation feature data and the third mutation feature data into a pre-trained target mutation detection model and outputting a mutation detection result of each suspected mutation site.
